(19) United States
(12) Patent Application Publication
(10) Pub. No.: US 2022/0328135 A1
(43) Pub. Date: Oct. 13, 2022

(54) BASE MODIFICATION ANALYSIS USING ELECTRICAL SIGNALS

(71) Applicant: The Chinese University of Hong Kong, Shatin (HK)

(72) Inventors: Yuk-Ming Dennis Lo, Hong Kong SAR (CN); Rossa Wai Kwun Chiu, Hong Kong SAR (CN); Kwan Chee Chan, Hong Kong SAR (CN); Peiyong Jiang, Hong Kong SAR (CN); Suk Hang Cheng, Hong Kong SAR (CN); Jiaen Deng, Hong Kong SAR (CN)

(21) Appl. No.: 17/719,166

(22) Filed: Apr. 12, 2022

Related U.S. Application Data
(60) Provisional application No. 63/173,728, filed on Apr. 12, 2021.

(51) Int. Cl. G16B 40/20; G16B 40/10; G16B 30/00

(52) U.S. Cl. CPC G16B 40/20; G16B 40/10; G16B 30/00; G16B 20/10

(57) ABSTRACT

Systems and methods for determining base modifications using electrical signals and other data is described herein. Embodiments can make use of features derived from electrical signals related to sequencing, such as those acquired from using a nanopore, that are affected by the various base modifications, as well as an identity of nucleotides in a window around a target position whose methylation status is determined. Other features may include a vector of statistical values of a segment of the electrical signal corresponding to the nucleotide and a statistical value of the electrical signal in a window in a region of the nucleic acid molecule. The detected base modifications can be used for additional analysis of a biological sample.

### CROSS-REFERENCE TO RELATED APPLICATIONS

The present application claims the benefit of priority to U.S. Provisional Patent Application 63/173,728, filed on Apr. 12, 2021, which is hereby incorporated by reference in its entirety and for all purposes.

### BACKGROUND

The existence of base modifications in nucleic acids varies throughout different organisms including viruses, bacteria, plants, fungi, nematodes, insects, and vertebrates (e.g., humans), etc. The most common base modifications are the addition of a methyl group to different DNA bases at different positions, so-called methylation. Methylation has been found on cytosines, adenines, thymines and guanines, such as 5mC (5-methylcytosine), 4mC (N4-methylcytosine), 5hmC (5-hydroxymethylcytosine), 5fC (5-formylcytosine), 5caC (5-carboxylcytosine), 1mA (N1-methyladenine), 3mA (N3-methyladenine), N6-methyladenine (6mA), 7mA (N7-methyladenine), 3mC (N3-methylcytosine), 2mG (N2-methylguanine), 6mG (O6-methylguanine), 7mG (N7-methylguanine), 3mT (N3-methylthymine), and 4mT (O4-methylthymine). In vertebrate genomes, 5mC is the most common type of base methylation, followed by that for guanine (i.e., in the CpG context).

DNA methylation is essential for mammalian development and has notable roles in gene expression and silencing, embryonic development, transcription, chromatin structure, X chromosome inactivation, protection against activity of the repetitive elements, maintenance of genomic stability during mitosis, and the regulation of parent-of-origin genomic imprinting.

DNA methylation plays many important roles in the silencing of promoters and enhancers in a coordinated manner (Robertson, 2005; Smith and Meissner, 2013). Many human diseases have been found to be associated with aberrations of DNA methylation, including but not limited to imprinting disorders (e.g. Beckwith-Wiedemann syndrome and Prader-Willi syndrome), repeat-instability diseases (e.g. fragile X syndrome), autoimmune disorders (e.g. systemic lupus erythematosus), metabolic disorders (e.g. type I and type II diabetes), neurological disorders, aging, etc.

The accurate measurement of methylomic modification on DNA molecules would have numerous clinical implications. One widely used method to measure DNA methylation is through the use of bisulfite sequencing (BS-seq) (Lister et al., 2009; Frommer et al., 1992). In this approach, DNA samples are first treated with bisulfite which converts unmethylated cytosine (i.e. C) to uracil. In contrast, the methylated cytosine remains unchanged. The bisulfite modified DNA is then analyzed by DNA sequencing. In another approach, following bisulfite conversion, the modified DNA is then subjected to polymerase chain reaction (PCR) amplification using primers that can differentiate bisulfite converted DNA of different methylation profiles (Herman et al., 1996). This latter approach is called methylation-specific PCR.

One disadvantage of such bisulfite-based approaches is that the bisulfite conversion step has been reported to significantly degrade the majority of the treated DNA (Grunau, 2001). Another disadvantage is that the bisulfite conversion step would create strong CG biases (Olova et al., 2018), resulting in the reduction of signal-to-noise ratios typically for DNA mixtures with heterogeneous methylation states. Furthermore, bisulfite sequencing would not be an ideal method to sequence long DNA molecules because of the degradation of DNA during bisulfite treatment.

There are many continuing efforts in achieving bisulfite-free determination of base modifications of nucleic acids. However, there is a paucity of commercially-viable tools that have achieved sensitivity and specificity levels comparable to bisulfite sequencing. Nanopore sequencing is a type of sequencing that is attractive for not needing chemical labeling of a sample. Detection of base modifications with nanopore sequencing may be relatively low cost and efficient.

Thus, there is a need to determine base modifications with nanopore sequencing. In this disclosure, we describe new methods and systems to process the electrical current signals produced by nanopore sequencing with high sensitivity and specificity for base modification determination.

### BRIEF SUMMARY

Embodiments described allow the determination of base modifications, such as 5mC in nucleic acids without template DNA pre-treatment such as enzymatic and/or chemical conversions, or protein and/or antibody binding. The embodiments present in this disclosure could be used for detecting different types of base modification, for example, including but not limited to 4mC, 5hmC, 5fC, 5caC, 1mA, 3mA, 6mA, 7mA, 3mC, 2mG, 6mG, 7mG, 3mT, 4mT, etc. Such embodiments can make use of features derived from electrical signals related to sequencing, such as those acquired from using a nanopore, that are affected by the various base modifications, as well as an identity of nucleotides in a window around a target position whose methylation status is determined. The raw electrical signal for a nucleotide may also be related to nucleotides upstream or downstream of the nucleotide. The raw electrical signal may be assigned to different nucleotides using suitable techniques.

Embodiments of the present invention can be used with nanopore sequencing. One example of a nanopore sequencing system is that commercialized by Oxford Nanopore Technologies. Methods may use the electrical signal measured using the nanopore. Methods may use the identity of the nucleotide, a position of the nucleotide with respect to a target position, a vector including a statistical value of a segment of the electrical signal corresponding to the nucleotide, and a statistical value of the electrical signal in a window in a region of the nucleic acid molecule.

The methods we have developed can serve as tools to detect base modifications in biological samples to assess the methylation profiles in the samples for various purposes including but not limited to research and diagnostic purposes. The detected methylation profiles can be used for different analysis. The methylation profiles can be used to detect the origin of DNA (e.g., maternal or fetal, tissue, bacterial). Detection of aberrant methylation profiles in tissues aids the identification of developmental and other disorders in individuals.

A better understanding of the nature and advantages of embodiments of the present invention may be gained with reference to the following detailed description and the accompanying drawings.

### BRIEF DESCRIPTION OF THE DRAWINGS

FIG. 1 illustrates nanopore sequencing.

FIG. 2 illustrates different signal features according to embodiments of the present invention.

FIG. 3 illustrates electrical current signal segmentation and the construction of signal feature vector according to embodiments of the present invention.

FIG. 4 is a graph of the distribution of lengths (i.e., durations) of events for each nucleotide passing through a nanopore according to embodiments of the present invention.

FIG. 5 illustrates principles for 5mC detection using integrated presentation matrices comprising electrical current patterns, sequencing positions, and sequencing context according to embodiments of the present invention.

FIG. 6 illustrates principles for base modification detection using integrated presentation matrices comprising electrical current patterns, sequencing positions, and sequencing context on the basis of both stands of a double-stranded DNA according to embodiments of the present invention.

FIG. 7 shows the impact of kernel size on the performance of base modification analysis according to embodiments of the present invention.

FIG. 8 shows the number of sequencing molecules used for training and testing in terms of methylation detection according to embodiments of the present invention.

FIGS. 9A-9D are boxplots of the probability of being methylated for a CpG between WGA DNA and M.SssI-treated DNA datasets using IPM-CNN and IPM-RNN approaches according to embodiments of the present invention.

FIGS. 10A and 10B show receiver operator characteristic (ROC) curves for training dataset and testing dataset according to embodiments of the present invention.

FIG. 11 is a table of the performance of different tools for methylation analysis according to embodiments of the present invention.

FIG. 12 is a flowchart of a process of detecting a modification of a nucleotide in a nucleic acid molecule according to embodiments of the present invention.

FIG. 13 is a flowchart of a process of detecting a modification of a nucleotide in a nucleic acid molecule according to embodiments of the present invention.

FIG. 14 illustrates a measurement system according to embodiments of the present invention.

FIG. 15 shows a block diagram of an example computer system usable with systems and methods according to embodiments of the present invention.

FIG. 16 shows a graph of the effect of different combinations of parameters on area under the ROC curve (AUC) according to embodiments of the present invention.

FIG. 17 shows a graph of the effect of the window size on AUC according to embodiments of the present invention.

FIG. 18 illustrates principles for 6mA detection using integrated presentation matrices comprising electrical current patterns, sequencing positions, and sequencing context according to embodiments of the present invention.

FIG. 19 shows a graph of the AUC of 6mA detection according to embodiments of the present invention.

FIG. 20 is a comparison of single-molecule methylation levels determined by IPM-RNN model for DNA originating from buffy coat and NPC tumor samples according to embodiments of the present invention.

FIG. 21 shows examples of single-molecule methylation patterns according to embodiments of the present invention.

FIG. 22 is a graph of single-molecule methylation levels of maternal-specific and fetal-specific cell-free DNA molecules according to embodiments of the present invention.

FIG. 23 is an ROC curve for determining fetal and maternal origin of cell-free DNA molecules using methylation patterns determined by the IPM-CNN model according to embodiments of the present invention.

### TERMS

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus; tissues in a subject who has received transplantation; tissues of an organism that are infected by a microorganism or a virus) or to healthy cells vs. tumor cells. “Reference tissues” can correspond to tissues used to determine tissue-specific methylation levels. Multiple samples of a same tissue type from different individuals may be used to determine a tissue-specific methylation level for that tissue type.

A “biological sample” refers to any cellular sample that is taken from a human subject. The biological sample can be a tissue biopsy, a fine needle aspirate, or blood cells. The sample can also be a cell-free sample taken from a pregnant woman, for example, plasma or serum or urine. In various embodiments, the majority of DNA in a biological sample from a pregnant woman that has been enriched for cell-free DNA (e.g., a plasma sample obtained via a centrifugation protocol) can be cell-free, e.g., greater than 50%, 60%, 70%, 80%, 90%, 95%, or 99% of the DNA can be cell-free. The centrifugation protocol can include, for example, 3,000 g×10 minutes, obtaining the fluid part, and re-centrifuging at for example, 30,000 g for another 10 minutes to remove residual cells. In certain embodiments, following the 3,000 g centrifugation step, one can follow up with filtration of the fluid part (e.g. using a filter of pore size of 5 μm, or smaller, in diameter).

A “sequence read” refers to a string of nucleotides sequenced from any part or all of a nucleic acid molecule. For example, a sequence read may be a short string of nucleotides (e.g., 20-150) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

A “site” (also called a “genomic site”) corresponds to a single site, which may be a single base position or a group of correlated base positions, e.g., a CpG site or larger group of correlated base positions. A “locus” may correspond to a region that includes multiple sites. A locus can include just one site, which would make the locus equivalent to a site in that context.

A “methylation status” refers to the state of methylation at a given site. For example, a site may be either methylated, unmethylated, or in some cases, undetermined.

The “methylation index” for each genomic site (e.g., a CpG site) can refer to the proportion of DNA fragments (e.g., as determined from sequence reads or probes) showing methylation at the site over the total number of reads covering that site. A “read” can correspond to information (e.g., methylation status at a site) obtained from a DNA fragment. A read can be obtained using reagents (e.g. primers or probes) that preferentially hybridize to DNA fragments of a particular methylation status at one or more sites. Typically, such reagents are applied after treatment with a process that differentially modifies or differentially recognizes DNA molecules depending on their methylation status, e.g. bisulfite conversion, or methylation-sensitive restriction enzyme, or methylation binding proteins, or anti-methylcytosine antibodies, or single molecule sequencing techniques (e.g. single molecule, real-time sequencing (e.g., from Pacific Biosciences) and nanopore sequencing (e.g. from Oxford Nanopore Technologies)) that recognize methylcytosines and hydroxymethylcytosines.

The “methylation density” of a region can refer to the number of reads at sites within the region showing methylation divided by the total number of reads covering the sites in the region. The sites may have specific characteristics, e.g., being CpG sites. Thus, the “CpG methylation density” of a region can refer to the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of cytosines not converted after bisulfite treatment (which corresponds to methylated cytosine) at CpG sites as a proportion of all CpG sites covered by sequence reads mapped to the 100-kb region. This analysis can also be performed for other bin sizes, e.g. 500 bp, 5 kb, 10 kb, 50-kb or 1-Mb, etc. A region could be the entire genome or a chromosome or part of a chromosome (e.g. a chromosomal arm). Alternatively, the methylation density can be determined without bisulfite conversion using nanopore sequencing using embodiments described in this disclosure. The methylation index of a CpG site is the same as the methylation density for a region when the region only includes that CpG site. The “proportion of methylated cytosines” can refer the number of cytosine sites, “C's”, that are shown to be methylated (for example unconverted after bisulfite conversion) over the total number of analyzed cytosine residues, i.e. including cytosines outside of the CpG context, in the region. The methylation index, methylation density, count of molecules methylated at one or more sites, and proportion of molecules methylated (e.g., cytosines) at one or more sites are examples of “methylation levels.” Apart from bisulfite conversion, other processes known to those skilled in the art can be used to interrogate the methylation status of DNA molecules, including, but not limited to enzymes sensitive to the methylation status (e.g. methylation-sensitive restriction enzymes), methylation binding proteins, single molecule sequencing using a platform sensitive to the methylation status (e.g. nanopore sequencing (Schreiber et al. Proc Natl Acad Sci 2013; 110: 18910-18915) and by single molecule, real-time sequencing (e.g. that from Pacific Biosciences) (Flusberg et al. Nat Methods 2010; 7: 461-465)).

A “methylome” provides a measure of an amount of DNA methylation at a plurality of sites or loci in a genome. The methylome may correspond to all of the genome, a substantial part of the genome, or relatively small portion(s) of the genome.

A “pregnant plasma methylome” is the methylome determined from the plasma or serum of a pregnant animal (e.g., a human). The pregnant plasma methylome is an example of a cell-free methylome since plasma and serum include cell-free DNA. The pregnant plasma methylome is also an example of a mixed methylome since it is a mixture of DNA from different organs or tissues or cells within a body. In one embodiment, such cells are the hematopoietic cells, including, but not limited to cells of the erythroid (i.e. red cell) lineage, the myeloid lineage (e.g., neutrophils and their precursors), and the megakaryocytic lineage. In pregnancy, the plasma methylome may contain methylomic information from the fetus and the mother. The “cellular methylome” corresponds to the methylome determined from cells (e.g., blood cells) of the patient. The methylome of the blood cells is called the blood cell methylome.

A “methylation profile” includes information related to DNA or RNA methylation for multiple sites or regions. Information related to DNA methylation can include, but not limited to, a methylation index of a CpG site, a methylation density (MD for short) of CpG sites in a region, a distribution of CpG sites over a contiguous region, a pattern or level of methylation for each individual CpG site within a region that contains more than one CpG site, and non-CpG methylation. In one embodiment, the methylation profile can include the pattern of methylation or non-methylation of more than one type of base (e.g. cytosine or adenine). A methylation profile of a substantial part of the genome can be considered equivalent to the methylome. “DNA methylation” in mammalian genomes typically refers to the addition of a methyl group to the 5′ carbon of cytosine residues (i.e. 5-methylcytosines) among CpG dinucleotides. DNA methylation may occur in cytosines in other contexts, for example CHG and CHH, where H is adenine, cytosine or thymine. Cytosine methylation may also be in the form of 5-hydroxymethylcytosine. Non-cytosine methylation, such as N⁶-methyladenine, has also been reported.

A “methylation pattern” refers to the order of methylated and non-methylated bases. For example, the methylation pattern can be the order of methylated bases on a single DNA strand, a single double-stranded DNA molecule, or another type of nucleic acid molecule. As an example, three consecutive CpG sites may have any of the following methylation patterns: UUU, MMM, UMM, UMU, UUM, MUM, MUU, or MMU, where “U” indicates an unmethylated site and “M” indicates a methylated site. When one extends this concept to base modifications that include, but not restricted to methylation, one would use the term “modification pattern,” which refers to the order of modified and non-modified bases. For example, the modification pattern can be the order of modified bases on a single DNA strand, a single double-stranded DNA molecule, or another type of nucleic acid molecule. As an example, three consecutive potentially modifiable sites may have any of the following modification patterns: UUU, MMM, UMM, UMU, UUM, MUM, MUU, or MMU, where “U” indicates an unmodified site and “M” indicates a modified site. One example of base modification that is not based on methylation is oxidation changes, such as in 8-oxo-guanine.

The terms “hypermethylated” and “hypomethylated” may refer to the methylation density of a single DNA molecule as measured by its single molecule methylation level, e.g., the number of methylated bases or nucleotides within the molecule divided by the total number of methylatable bases or nucleotides within that molecule. A hypermethylated molecule is one in which the single molecule methylation level is at or above a threshold, which may be defined from application to application. The threshold may be 5%, 10%, 20%, 30%, 40%, 50%, 60%, 70%, 80%, 90%, or 95%. A hypomethylated molecule is one in which the single molecule methylation level is at or below a threshold, which may be defined from application to application, and which may change from application to application. The threshold may be 5%, 10%, 20%, 30%, 40%, 50%, 60%, 70%, 80%, 90%, or 95%.

The terms “hypermethylated” and “hypomethylated” may also refer to the methylation level of a population of DNA molecules as measured by the multiple molecule methylation levels of these molecules. A hypermethylated population of molecules is one in which the multiple molecule methylation level is at or above a threshold which may be defined from application to application, and which may change from application to application. The threshold may be 5%, 10%, 20%, 30%, 40%, 50%, 60%, 70%, 80%, 90%, or 95%. A hypomethylated population of molecules is one in which the multiple molecule methylation level is at or below a threshold which may be defined from application to application. The threshold may be 5%, 10%, 20%, 30%, 40%, 50%, 60%, 70%, 80%, 90%, and 95%. In one embodiment, the population of molecules may be aligned to one or more selected genomic regions. In one embodiment, the selected genomic region(s) may be related to a disease such as a genetic disorder, an imprinting disorder, an epigenetic disorder, a metabolic disorder, or a neurological disorder. The selected genomic region(s) can have a length of 50 nucleotides (nt), 100 nt, 200 nt, 300 nt, 500 nt, 1000 nt, 2 knt, 5 knt, 10 knt, 20 knt, 30 knt, 40 knt, 50 knt, 60 knt, 70 knt, 80 knt, 90 knt, 100 knt, 200 knt, 300 knt, 400 knt, 500 knt, or 1 Mnt.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1).

The terms “cutoff” and “threshold” refer to predetermined numbers used in an operation. For example, a cutoff size can refer to a size above which fragments are excluded. A threshold value may be a value above or below which a particular classification applies. Either of these terms can be used in either of these contexts. A cutoff or threshold may be “a reference value” or derived from a reference value that is representative of a particular classification or discriminates between two or more classifications. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical analyses or simulations of samples.

A “level of pathology” (or level of disorder) can refer to the amount, degree, or severity of pathology associated with an organism that can be measured through analysis of its cells. Another example of pathology is a rejection of a transplanted organ. Other example pathologies can include genomic imprinting disorders, autoimmune attack (e.g., lupus nephritis damaging the kidney or multiple sclerosis damaging the nervous system), inflammatory diseases (e.g., hepatitis), fibrotic processes (e.g. cirrhosis), fatty infiltration (e.g. fatty liver diseases), degenerative processes (e.g. Alzheimer's disease), and ischemic tissue damage (e.g., myocardial infarction or stroke). A healthy state of a subject can be considered a classification of no pathology.

A “pregnancy-associated disorder” include any disorder characterized by abnormal relative expression levels of genes in maternal and/or fetal tissue. These disorders include, but are not limited to, preeclampsia, intrauterine growth restriction, invasive placentation, pre-term birth, hemolytic disease of the newborn, placental insufficiency, hydrops fetalis, fetal malformation, HELLP (hemolysis, elevated liver enzymes, and a low platelet count) syndrome, systemic lupus erythematosus (SLE), and other immunological diseases of the mother. In some embodiments, a pregnancy-associated disorder is any condition associated with physiological or morphological aberrations during the pregnancy period.

The abbreviation “bp” refers to base pairs. In some instances, “bp” may be used to denote a length of a DNA fragment, even though the DNA fragment may be single stranded and does not include a base pair. In the context of single-stranded DNA, “bp” may be interpreted as providing the length in nucleotides.

The abbreviation “nt” refers to nucleotides. In some instances, “nt” may be used to denote a length of a single-stranded DNA in a base unit. Also, “nt” may be used to denote the relative positions such as upstream or downstream of the locus being analyzed. In some contexts concerning technological conceptualization, data presentation, processing and analysis, “nt” and “bp” may be used interchangeably.

The term “sequence context” can refer to the base compositions (A, C, G, or T) and the base orders in a stretch of DNA. Such a stretch of DNA could be surrounding a base that is subjected to or the target of base modification analysis. For example, the sequence context can refer to bases upstream and/or downstream of a base that is subjected to base modification analysis.

The term “machine learning models” may include models based on using sample data (e.g., training data) to make predictions on test data, and thus may include supervised learning. Machine learning models often are developed using a computer or a processor. Machine learning models may include statistical models.

The term “data analysis framework” may include algorithms and/or models that can take data as an input and then output a predicted result. Examples of “data analysis frameworks” include statistical models, mathematical models, machine learning models, other artificial intelligence models, and combinations thereof.

The term “real-time sequencing” may refer to a technique that involves data collection or monitoring during a process involved in sequencing. For example, real-time sequencing may involve electrical signal monitoring of ionic current through a nanopore when a nucleotide strand translocating that nanopore.

The term “electrical signal” may refer to a voltage or current that conveys information. The electrical signal could be expressed in a variety of regular and/or irregular signal waveform types and/or shapes such as square waves, rectangular waves, triangular waves, saw-toothed waveforms, or a variety of pulses and spikes. Electrical signal may include visual representations of variations of a voltage or current over time. The measurement of electrical signal could be sampled at particular times (e.g., millisecond). For example, the electrical current is sampled at a frequency of 1 kHz, 2 kHz, 3 kHz, 4 kHz, 5 kHz, 10 kHz, 20 kHz, 30 kHz, 40 kHz, 50 kHz, 100 kHz, etc.

The term “signal segment” or “segment” may refer to a portion of the trace of an electrical signal associated with sequencing a particular nucleotide. The segment may correspond to the nucleotide determined from base-calling in nanopore sequencing. The segment may cover a certain duration of the trace. Different segments may have different durations. Segments may be non-overlapping. In some embodiments, the electrical signal amplitude may have a certain variation in the segment. For example, the electrical signal amplitude may be within 5%, 10%, 20%, 30%, or 40% of the mean or median electrical signal amplitude in the segment.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

### DETAILED DESCRIPTION

Accurate and efficient methods of detecting base modifications (e.g., methylation) using nanopore sequencing are desired. Research studies have studied the feasibility of using electrical signals produced by nanopore sequencing for analyzing DNA methylation (Simpson et al. Nat Methods. 2017; 14:407-410; Liu et al. Nat Commun. 2019; 10:2449; Ni et al. Bioinformatics. 2019; 35:4586-4595). The reported performance for 5-methylcytosine (5mC) detection was suboptimal in many validation studies. For instance, the sensitivity of 5mC detection using a computational tool, named DeepSignal, was reported to be 79% with a specificity of 88% when analyzing H. sapiens R9.4 1D data based on sample NA12878 (Ni et al. Bioinformatics. 2019; 35:4586-4595). If one aimed to achieve a higher specificity (e.g. >95%), the sensitivity would be expected to further deteriorate. For another tool, called nanopolish (Liu et al. Nat Commun. 2019; 10:2449), when analyzing the same dataset, the sensitivity was only 0.61 with a specificity of 0.46. The nanopolish software was based on the hidden Markov model with the following assumptions: (1) the electrical signals of a 6-nucleotide oligomer (i.e. 6-mer) in a DNA sequence followed Gaussian distributions; (2) the probability of a methylation state (methylation or unmethylation) for a particular base depended only on the methylation state of the previous base; (3) the probability of output a particular electrical current level depended only on the methylation state that produced the electrical current signals and not on any other methylation states or any other electrical current signals. Those assumptions might be incorrect in real electrical current signals produced during nanopore sequencing, thus leading to lower sensitivity and specificity.

A recent computational tool, named DeepMod, for DNA methylation analysis based on Oxford Nanopore sequencing, attempted to use a bidirectional recurrent neural network (RNN). However, the design of such an approach aimed to measure methylation levels in a genomic position by aggregating the prediction results from sequencing reads with electrical signals, thus lacking the ability to analyze the methylation patterns at a single-molecule level. In addition, the median sequencing depth across datasets, including Escherichia coli, Chlamydomonas reinhardtii, and Homo sapiens, was approximately 33×. In many commercial applications, lower sequencing depth would be desirable to save economic costs and analysis times. It is unknown whether the DeepMod software would be capable of analyzing the methylation patterns with a practically meaningful accuracy at a single-molecule level.

In one study, Yuen et al. systematically benchmarked tools for CpG methylation detection from nanopore sequencing, and concluded that most tools showed high dispersion and low agreement with the expected percentage methylation per CpG site (Yuen et al. bioRxiv. 2020; doi: doi.org/10.1101/2020.10.14.340315).

Tse et al. reported using single molecule real-time sequencing (SMRT-seq) from Pacific Biosciences (PacBio), the kinetic features of DNA polymerase, including optical signals such as interpulse durations (IPDs) and pulse widths (PWs) produced by the incorporation of fluorophore-labeled nucleotides during DNA polymerization, could be used for differentiating the methylated and unmethylated CpG sites, on the basis of analyzing measurement window consisting of more than one base with the use of the convolutional neural network (Tse et al. Proc Natl Acad Sci USA. 2021; 118: e2019768118; U.S. Pat. No. 11,091,794). Such a measurement window organized IPDs and PWs into different sequencing contexts and sequencing positions. However, nanopore sequencing used a completely different sequencing mechanism, depending on the electrical current signals caused by a strand of double-stranded DNA passing through a nanopore. Such raw electrical signals vary according to different nucleotides passing through a nanopore, and the electrical signals of particular nucleotide would be affected by upstream and downstream nucleotides nearby that nucleotide. Hence, different nucleotides would have different lengths of electrical signal traces detected, and even identical nucleotides would have different lengths of electrical signal traces. When analyzing the electrical signals associated with a particular nucleotide or more than one nucleotide that passes through a nanopore, the lengths of electrical signal traces detected on each base is not fixed over time. In contrast, the previous study for 5mC detection using PacBio SMRT-seq was based on two fixed measurements related to optical signals for each nucleotide, namely IPD and PW (Tse et al. Proc Natl Acad Sci USA. 2021; 118: e2019768118). Hence, the trained models presented in Tse et al.'s study (Tse et al. Proc Natl Acad Sci USA. 2021; 118: e2019768118) are not applicable to such electrical signals produced by nanopore sequencing.

Embodiments described herein use electrical signals obtained from nanopore sequencing to detect nucleotide modifications. The nucleotide modification may include any methylation described herein. Information obtained from nanopore sequencing may include the identity of the nucleotide, a position of the nucleotide with respect to a target position, a vector including a statistical value of a segment of the electrical signal corresponding to the nucleotide, and a statistical value of the electrical signal in a window in a region of the nucleic acid molecule.

The embodiments present in this disclosure can be used for DNA obtained from cellular samples obtained from an organism (e.g., cell lines, solid organs, solid tissues, a sample obtained via endoscopy, chorionic villus sample). The embodiments in this disclosure can also be used for cellular samples obtained from the environment (e.g. bacteria, cellular contaminants), food (e.g., meat). The embodiments present in this disclosure can also be used for plasma or serum obtained from a pregnant woman. In some embodiments, the methods present in this disclosure can also be applied following a step in which a fraction of the genome is first enriched, e.g. using hybridization probes (Albert et al., 2007; Okou et al., 2007; Lee et al., 2011), or approaches based on physical separation (e.g. based on sizes, etc) or following restriction enzyme digestion (e.g. MspI), or Cas9-based enrichment (Watson et al., 2019). While the invention does not require enzymatic or chemical conversion to work, in certain embodiments, such a conversion step can be included to further enhance the performance of the invention.

Embodiments of the present disclosure improve nanopore sequencing to be able to detect modified bases accurately and efficiently. The base modification may be detected directly. Embodiments may avoid enzymatic or chemical conversion, which may not preserve all modification information for detection. Additionally, certain enzymatic or chemical conversions may not be compatible with certain types of modifications. Embodiments of the present disclosure may also avoid amplification by PCR, which may not transfer base-modification information to the PCR products. Additionally, both strands of DNA may be sequenced together, thereby enabling the pairing of the sequence from one strand with its complementary sequence to the other strand. By contrast, PCR amplification splits the two strands of double-stranded DNA, so such combined analysis of sequences from the two constituent strands is difficult.

Furthermore, nanopore sequencing is more cost-effective and portable than other sequencing techniques. For instance, a nanopore sequencing system, Oxford Nanopore Technologies MinION™ is approximately 5,000 USD, while an optical-signal based sequencing system, PacBio SMRT™ Sequel II system, is on the order of 500,000 to 700,000 USD. Nanopore sequencing speeds are at about 450 nucleotides per second, while PacBio SMRT™ sequencing is about 5 nucleotides per second. Hence, within the same time period, nanopore sequencing can obtain more data than with an optical-signal based sequencing system.

Methylation profiles, determined with or without enzymatic or chemical conversion, can be used for analyzing biological samples. In one embodiment, the methylation profiles can be used to detect the origin of cellular DNA (e.g., maternal or fetal, tissue, or viral). Detection of aberrant methylation profiles in tissues aid the identification of developmental disorders in individuals. Methylation patterns in a single molecule can identify chimeric (e.g., between a virus and human) and hybrid DNA, (e.g., between two genes normally unfused in a natural genome); or between two species (e.g., through genetic or genomic manipulation).

### I. NANOPORE SEQUENCING PRINCIPLES

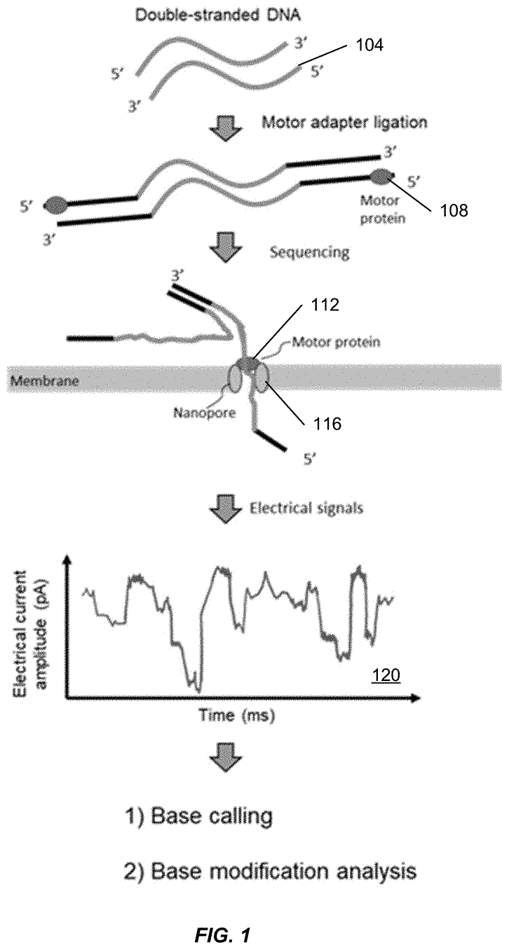

An example of single-molecule sequencing technology is nanopore sequencing (Oxford Nanopore Technologies). FIG. 1 shows the principle for nanopore sequencing of DNA molecules (e.g., DNA molecule 104). The electrical signal patterns caused by the ionic current flows across a membrane was used for determining the sequence of nucleic acids, as a single DNA molecule passes through a pore of nanometer size. Such a pore may, for example, but not limited to, be constructed by a protein (e.g. alpha-hemolysin, aerolysin, and Mycobacterium smegmatis porin A (MspA)) or synthetic materials such as silicon or graphene (Magi et al, Brief Bioinform. 2018; 19:1256-1272).

In one embodiment, double-stranded DNA molecules were subjected to an end-repair process. Such a process would convert DNA into blunt-end DNA, followed by the addition of an A tail that facilitated sequencing adaptor ligation. Sequencing adapters each carrying a motor protein (i.e., motor adapter) (e.g., motor protein 108) are ligated to both ends of a DNA molecule. The process of sequencing starts as the motor protein (e.g., motor protein 112) unwinds a double-stranded DNA, enabling a first strand to pass through the nanopore. When the DNA strand passes through nanopore 116, a sensor (e.g., electrode) measures the ionic current changes in a picoampere (pA) over time (millisecond, ms), depending on the sequence contexts as well as the associated base modifications (called one-dimension (1D) read). Graph 120 shows an example current signal versus time. In another embodiment, hairpin sequence adaptors would be used for covalently tethering a first strand and its complementary strand together for a double-stranded DNA molecule. Hence, during sequencing, a strand of a double-stranded DNA molecule was sequenced, followed by the complementary strand (called 1D² or two-dimension (2D) read), which could potentially improve the sequencing accuracy. In yet another embodiment, one end of a double-strand DNA molecule tethered by a protein would increase the likelihood of sequencing the complementary strand that follows the completion of sequencing the first strand of the same molecule, generating 1D² reads.

Raw signals (e.g., current in graph 120) are used for base calling and base modification analyses. In some embodiments, the base calling and base modification analyses are conducted by means of a machine learning approach, for example, but not limited to, recurrent neural network (RNN), convolutional neural network (CNN), hidden Markov model (HMM), or their one or more combinations.

In one embodiment, we developed a new method to process the electrical current signals produced by nanopore sequencing, and the processed signals were analyzed for the determination of DNA methylation at a single molecule level, based on convolutional neural network (CNN) or recurrent neural network (RNN).

### II. ELECTRICAL CURRENT SIGNAL ANALYSIS

The electrical current signals from nanopore sequencing may be analyzed to identify base modifications. However, the machine learning approaches described in FIG. 1 do not use only an input of the raw current obtained using the nanopore. Embodiments described herein use one or more statistical values of portions of the electrical current. A vector of these one or more statistical values may be combined with other information corresponding to a window of nucleotides, including the identity of the nucleotide and a position of the nucleotide. The position of the nucleotide may be with respect to a target position within the window, where the target position is the position where the modification or lack thereof is detected. The information for the window of nucleotides may be included with a statistical value of the electrical signal in a region of the nucleic acid molecule to form an input data structure. Models trained on these input data structures can be used to detect base modifications.

A. Electrical Current Vector Parameters

For a nucleotide strand passing through a nanopore, one would detect N events (i.e., signal segments associated with different nucleotides identified). In one embodiment, one event corresponds to one nucleotide identified during base calling, with a series of electrical signals sampled at a particular unit in time (e.g. millisecond). In one example, the electrical current was sampled at a frequency of 4 kHz (Rang et al. Genome Biol. 2018; 19:90). In another embodiment, one event corresponds to more than one nucleotide identified during base calling, with a series of electrical signals sampled at a particular rate in time.

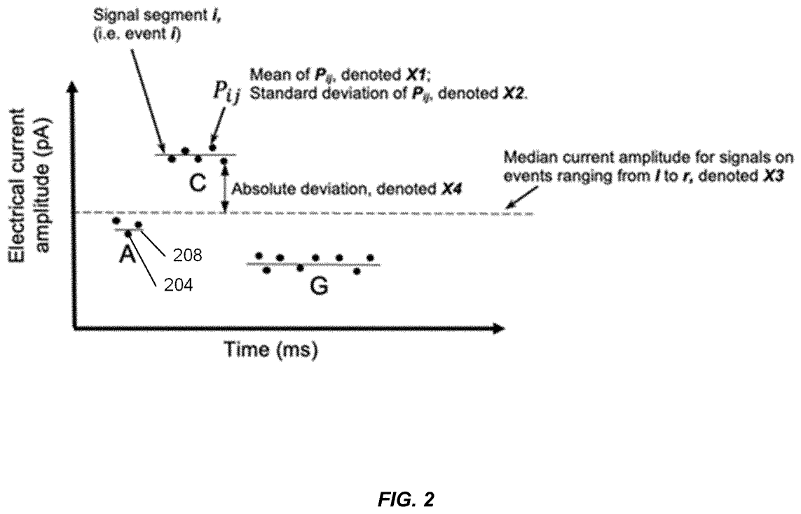

FIG. 2 shows a graph of electrical current signals. The electrical current amplitude in picoamperes on the y-axis. The time in milliseconds is on the x-axis. The dots (e.g., dot 204) show an individual signal measurement. A line through adjacent dots (e.g., line 208) indicates a signal segment of signal measurements associated with a nucleotide (e.g., A for line 208). For event i, assuming that there are mᵢ electrical current signals, the amplitude of an electrical current signal j on the event i was denoted by Pᵢⱼ. In one embodiment, for a nucleotide, a signal feature vector, including X1, X2, X3, X4, and X5, is used to characterize the pattern of electrical signals associated with that nucleotide. The definitions for X1, X2, and X3 are illustrated in FIG. 2. X1 is the mean of Pᵢⱼ. X2 is the standard deviation of Pᵢⱼ. X3 is the median of Pᵢⱼ. X4 is the median of the absolute deviations of current from X3 (only one absolute deviation labeled in FIG. 2). X5 is the difference of X1 from a mean of current signals divided by the standard deviation. X5 may be considered a z-score of the current signal of a segment.

In one embodiment, Pᵢⱼ could be a normalized signal. Normalization could involve rescaling the current signals from the original range so that the normalized signal values are within the range of 0 and 1, with the use of the minimum and maximum values concerning the part or whole nucleotide strand. Normalization could involve rescaling current signals so that the mean of normalized signal values is 0 and the standard deviation is 1. Normalization could involve rescaling the current signals with the use of median value and deviations concerning the part or whole nucleotide strand.

X1 and X2 represent the mean and standard deviation of Piᵢⱼ associated with event i.

X1 is defined by:

$$X 1 = \frac{{Σ}_{j = 1}^{j = {m}_{i}} {P}_{i j}}{{m}_{i}}$$

X2 is defined by:

$$X 2 = \sqrt{\frac{{{Σ}_{j = 1}^{j = {m}_{i}} ( {P}_{ij} - X 1 )}^{2}}{{m}_{i} - 1}}$$

X3 is defined by:

X3=median (Pᵢⱼ),

where i is ranged from l to r, including the events surrounding the base of inquiry for base modification analysis (e.g., methylation at a CpG site). The variables l and r represent the left and right of a window of a sequence of events (corresponding to a nucleotide sequence). A nucleotide sequence between l and r should generally be longer than the integrated presentation matrix (referred to as IPM) of current signal patterns discussed below. For a given event i,j is ranged from 1 to mᵢ. X3 may be the median current signal used in determining all segments. X3 may be the same value for all segments because X3 is determined using currents for more than just a single segment. In some embodiments, X3 may be for a particular window. In other embodiments, X3 may be a median spanning multiple windows.

X4 is defined by:

X4=median (|Pᵢⱼ−X3|),

where |·| represents the absolute value; and i is ranged from l to r, including the events surrounding the base of inquiry for base modification analysis (e.g. methylation at a CpG site). For a given i,j is ranged from 1 to mᵢ. X4 may be the median of the absolute deviation of the current signals used in determining all segments. X4 may be calculated using currents for more than just a single segment (e.g., using all sampled current values) and may therefore be the same value for all segments.

X5 is defined by:

$$X 5 = \frac{X 1 - μ}{σ} ,$$

$$where μ = \frac{{Σ}_{i = l}^{i = r} {Σ}_{j = 1}^{j = {m}_{r}} {P}_{ij}}{M - 1} , and$$

$$σ = \sqrt{\frac{{Σ}_{i = l}^{i = r} {{Σ}_{j = 1}^{j = {m}_{r}} ( {P}_{ij} - μ )}^{2}}{M - 1} ,}$$

wherein i is ranged from l to r, including the events surrounding the base of inquiry for base modification analysis (e.g. methylation at a CpG site). For a given i, j is ranged from 1 to mᵢ. M is the total number of current signals sampled for events ranging from l to r. The size of a region, which is associated with a plurality of electric current signals and used for determining X3, may be the size of a DNA fragment. For example, if a DNA fragment is 500 bp, then the size of the region is 500. If a fragment is 300 bp, then the size of region is 300. In some embodiments, it may be useful to further divide a DNA fragment into smaller sub-fragments for determining X3. The size of a region used for determining X3 may be 5 nt, 10 nt, 20 nt, 30 nt, 40 nt, 50 nt, 60 nt, 70 nt, 90 nt, 100 nt, 200 nt, 300 nt, 400 nt, 500 nt, 600 nt, 800 nt, 900 nt, 1 kb, 2 kb, 3 kb, 4 kb, 5 kb, 10 kb, 50 kb, etc.

X1 and X2 could be used for reflecting the signal changes within an event i, representing the local pattern of the electrical signals for each nucleotide. X3, X4, and X5 could be used for reflecting the signal changes for an event i relative to other surrounding events ranging from l to r. In some embodiments, surrounding events could be X-nt upstream and Y-nt downstream of the base of inquiry for base modification analysis. X could include, but is not limited to, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 300, 400, 500, 1000, 2000, 4000, 5000, and 10000; Y could include, but was not limited to, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 300, 400, 500, 1000, 2000, 4000, 5000, and 10000. In one embodiment, the surrounding events could be the whole nucleotide strand passing through a nanopore.

B. Single-Stranded Analysis

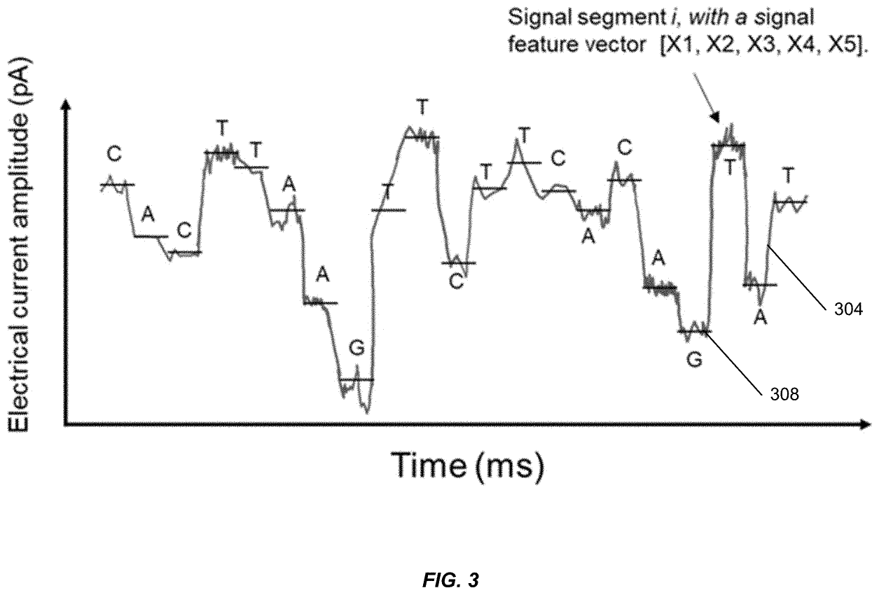

FIG. 3 shows a graph of electrical current signals. The electrical current amplitude in picoamperes on the y-axis. The time in milliseconds is on the x-axis. Trace 304 is the electrical current amplitude over time. Signal segments (e.g., segment 308) are parts of trace 304 that are associated with nucleotides. The electrical current changes would be varied according to the different nucleotides passing through the nanopore. Base-calling in nanopore sequencing generally relies on transforming current signals into different locally stationary status (i.e., events). The process of transforming current signals into different events is called electrical signal segmentation. The ionic current changes include, but not limited to, the amplitudes of events (e.g., measured in picoampere, pA) corresponding to one or more nucleotides in a signal segment, the direction of the ionic current, the duration of current events corresponding to one or more nucleotides in a signal segment, the rate of change of the ionic current, and the relative amplitudes across different signal segments. The amplitude may refer to the strength or magnitude of a current and need not imply an alternating current. Those current events are assigned to different bases using for example, a software, named Tombo (Stoiber et al bioRxiv. 2016; doi.org/10.1101/094672). One nucleotide would be associated with a series of events with different amplitudes. Such a tool (Tombo) attempted to test the difference in nanopore signals assigned to a genomic base between two samples to infer whether such a base was modified or not, on the basis of the Mann-Whitney U-test (Stoiber et al bioRxiv. 2016; doi.org/10.1101/094672). This tool (Tombo) did not take into account the upstream and downstream signals and sequence context and was not able to analyze the methylation patterns at a single molecule level as all signals from different sequence reads were aggregated into genomic bases. The performance of Tombo has been compared with those of other tools such as Nanopolish and DeepSignal (Yuen et al. bioRxiv. 2020; doi: doi.org/10.1101/2020.10.14.340315).

In one embodiment, to characterize the electrical current patterns within a signal segment related to a nucleotide, the mean (X1) and standard deviation (X2) of those electrical current amplitudes of events within that signal segment are calculated. The median value of current amplitudes of events associated with a whole molecule (X3) and the median absolute deviation of current amplitudes of events associated with a whole molecule (X4) is determined. The normalized signal (X5) for a signal segment is determined by the formula below:

$$X 5 = \frac{X1 - μ}{σ} ,$$

where X1 is the mean of those electrical current amplitudes of events within that signal segment related to a nucleotide in question; μ is the mean of those electrical current amplitudes of events within a whole molecule under investigation; σ is the standard deviation of those electrical current amplitudes of events within a whole molecule under investigation. In one embodiment, the mean and standard deviations could be derived after the removal of a small designated percentage of the largest and smallest values.

For a nucleotide, a signal feature vector, including X1, X2, X3, X4, and X5, is used to reflect the pattern of electrical signals associated with that nucleotide. For example, segment 308 may have a signal feature vector of [X1, X2, X3, X4, X5].

X1 and X2 represent the mean and standard deviation of electrical current amplitudes of events within a signal segment i. X3 represents the median value of current amplitudes of events associated with a whole molecule. X4 represents the median absolute deviation of current amplitudes of events associated with a whole molecule. X5 represents the normalized signal for a signal segment i.

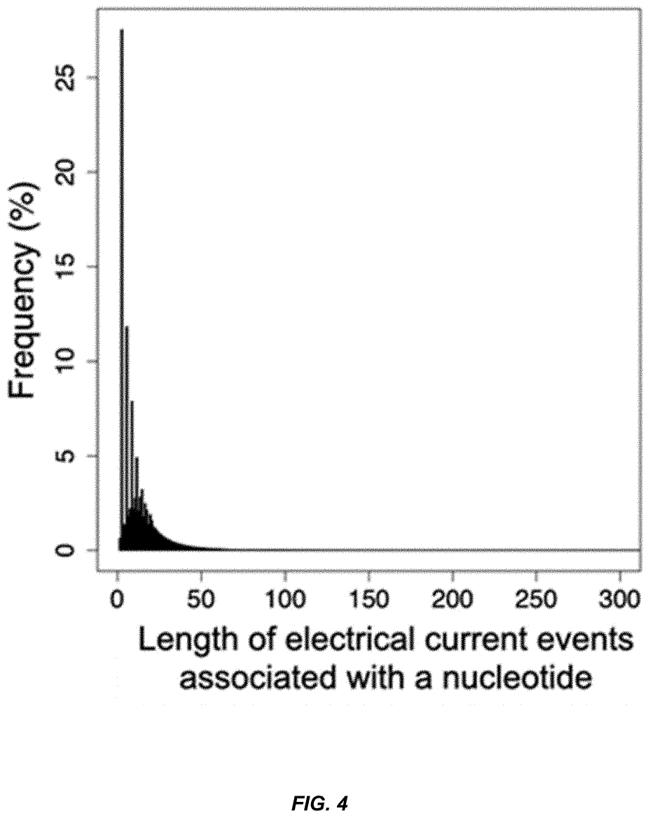

FIG. 4 is a plot of the frequencies of lengths of signal segments. The lengths (i.e., durations in milliseconds) of electrical current events associated with a nucleotide is on the x-axis. The frequency of the lengths is shown on the y-axis. FIG. 4 shows that the length of each signal segment associated with a nucleotide was variable with a median of 9 (range: 1-3540).

A base modification would affect the electrical signals associated with its upstream and downstream nucleotides. In this disclosure, we collectively made use of the electrical current signals related to a nucleotide for base modification analysis, the electrical current signals associated with nucleotides nearby the nucleotide of interest, as well as the sequencing context, so as to improve the performance. The DNA methylation at a CpG site (i.e., methylation at the 5^{th} carbon of cytosine) is the most common type of base methylation in vertebrate genomes. The analysis of DNA methylation at a CpG site was used as an illustrative example for this disclosure.

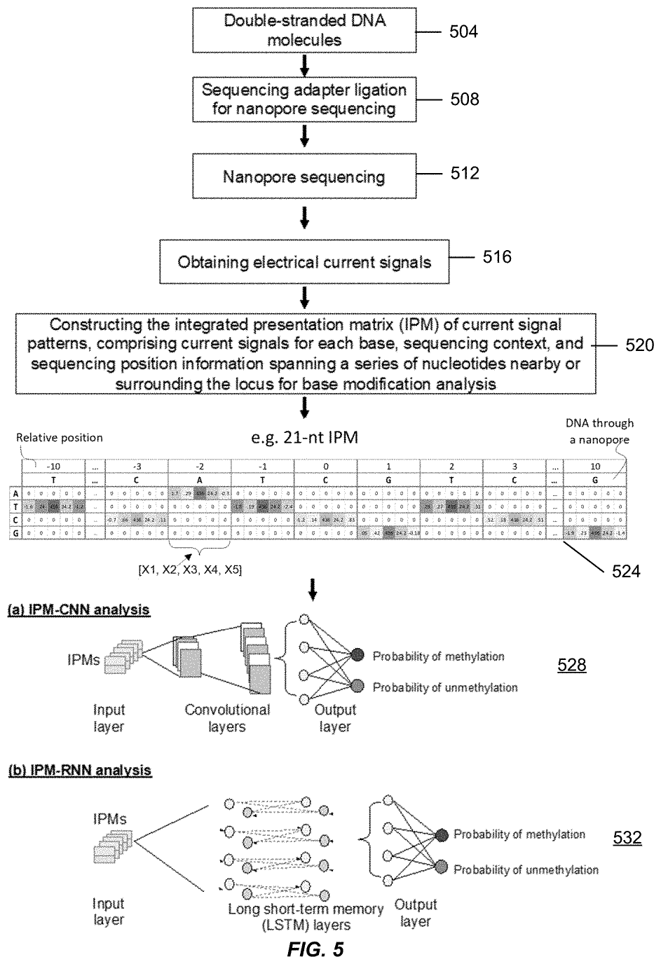

FIG. 5 shows the process for determining methylation using current signals from one strand through nanopore sequencing. At block 504, double-stranded DNA molecules are provided. At block 508, a double-stranded DNA molecule is ligated with sequencing adaptors, being suitable for nanopore sequencing. At block 512, nanopore sequencing is performed. A strand of a single double-stranded molecule moved through a pore embedded in a membrane, altering the ionic current signals that flow through the nanopore. At block 516, electrical current signals are obtained. The ionic current signals could be measured, for example, by a trans-electrode.

The current signals would be processed by a segmentation step using, for example, Tombo (Stoiber et al bioRxiv. 2016; doi.org/10.1101/094672). These segmented electrical events would be assigned to different nucleotides. At block 520, an integrated presentation matrix (IPM) is constructed. The IPM is a matrix of current signal patterns, which includes current signals for each base, sequencing context, and sequencing position information spanning a series of nucleotides nearby or surrounding the locus for base modification analysis. In one embodiment, the segmented electrical events associated with a nucleotide were described by a signal feature vector, i.e., [X1, X2, X3, X4, X5]. A cytosine within a CpG site and the, for example, 10-nt upstream and downstream of that cytosine (i.e., for example, a total of 21 nt), with a number of signal feature vectors, were used to form the IPM of current signal patterns. For illustration purposes, a 21-nt sequence of 5′-T[CCATGC]CATCGTC[GATGCA]G-3′ was used as an example, resulting in IPM 524. The bases in the brackets were left out (denoted by “. . . ”) for the sake of simplicity. For a position of −2 corresponding to the base of adenine (“A”), the signal feature vector associated with “A”, [X1=1.7, X2=0.29, X3=24.2, X4=436, X5=−0.3], was filled in the corresponding cells between a column of “−2” and a row of “A”. The other cells in the same columns were filled by “0”. The remaining signal feature vector for each nucleotide related to the 21-nt sequence context was filled using the same rule, thus forming a 21-nt IPM. Hence, such an IPM would simultaneously encode the current signal patterns, sequencing context, sequencing positions as well as the patterns changing over time. A number of IPMs originating from methylated and unmethylated DNA datasets were used for training a CNN or RNN model that was subsequently used to determine methylation status at a CpG site in test samples.

Block 528 shows CNN analysis. For CNN analysis, IPMs were fed into the input layer, followed by the process of convolutional layers and the output layer. The probability of methylation (i.e., output methylation score, range from 0 to 1) for a CpG was determined on the basis of a sigmoid function in the output layer. This approach was referred to as IPM-CNN. In one embodiment, the IPMs for methylated CpG sites (the M.SssI-treated DNA) and unmethylated CpG sites (the whole genome amplified (WGA) DNA) were used for training a CNN model. The target value of methylation for a CpG site in the dataset derived from M.Sss-treated DNA was defined as “1”, whereas the target value of methylation for a CpG site in the dataset derived from WGA DNA was defined as “0”. The optimal parameters of the IPM-CNN were obtained by minimizing the overall prediction error between the output scores calculated by a sigmoid function and desired target outputs (binary values: 0 or 1), through iteratively updating model parameters. The overall prediction error was determined by the sigmoid cross-entropy loss function in deep learning algorithms (keras.io/). The model parameters learned from the training datasets were used for analyzing methylation status in a testing dataset, outputting a probabilistic score (i.e. probability of methylation) suggesting the likelihood of a CpG site being methylated. In one embodiment, the CNN model made use of four two-dimensional (2D)-convolutional layers, each having 32, 64, 128, 256 filters with a kernel size of 25. The activation function of the rectified linear unit (ReLU) was used for those convolutional layers. A batch normalization layer was applied subsequently. A flattened layer was further added, followed by a dropout layer with a dropout rate of 0.5 and then followed by a fully connected layer comprising 200 neurons with the use of the ReLU activation function. The output layer with one neuron was finally applied, with a sigmoid activation function to yield the probabilistic score for a CpG site of being methylated (i.e. probability of methylation). The program for the CNN model was implemented on the basis of the Keras deep learning framework (https://keras.io/).

Block 532 shows RNN analysis. For RNN analysis, IPMs were fed into the input layer, followed by the process of long short-term memory (LSTM) layers and the output layer. The probability of methylation (range from 0 to 1) for a CpG was determined on the basis of a sigmoid function in the output layer. This approach was referred to as IPM-RNN. Using a training procedure similar to that used in IPM-RNN the optimal parameters of the IPM-RNN were obtained by minimizing the overall prediction error between the output scores calculated by a sigmoid function and desired target outputs (binary values: 0 or 1), through iteratively updating model parameters. The model parameters learned from the training datasets were used for analyzing methylation status in a testing dataset, outputting a probabilistic score (i.e. probability of methylation) suggesting the likelihood of a CpG site being methylated. In one embodiment, the RNN model with LSTM units was used with two full connection hidden layers, each having 256 hidden nodes. The last layer was followed by a dropout layer with a dropout rate 0.2. The output layer with one neuron was finally applied, with a sigmoid activation function to yield the probabilistic score for a CpG site of being methylated (i.e., probability of methylation). The program for the CNN model was implemented on the basis of the Keras deep learning framework (keras.io/).

C. Double-Stranded Analysis

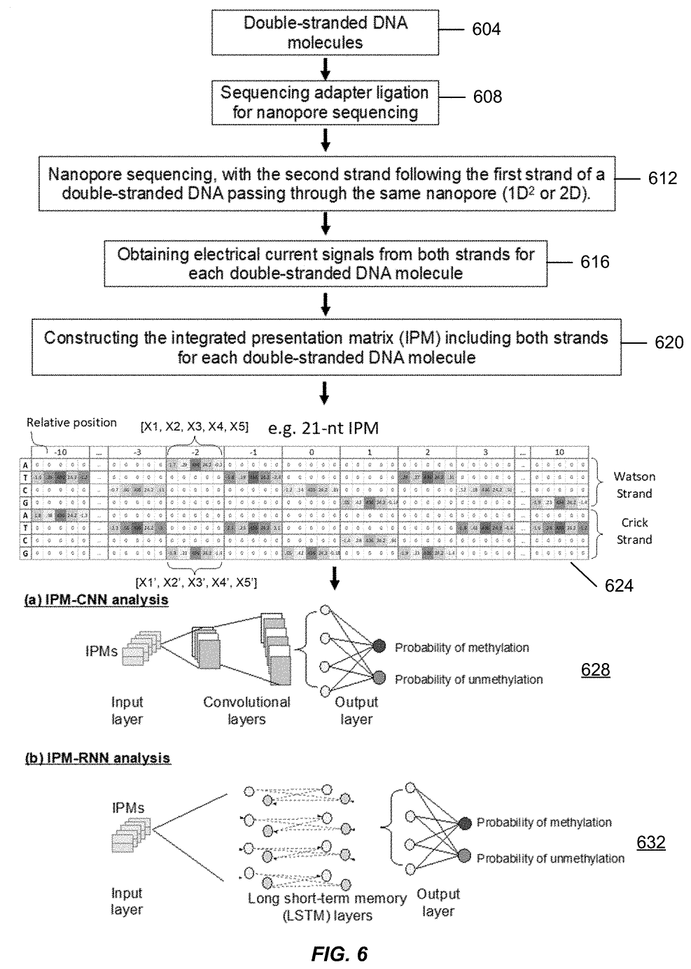

FIG. 6 shows the process for determining methylation using current signals from both DNA strands through nanopore sequencing. In one embodiment, the electrical current signals from both nucleotide strands of a double-stranded DNA molecule could be obtained, when such a double-stranded DNA molecule is sequenced in the way that the second nucleotide strand (referred to as the complementary strand, or the Crick strand) will immediately follow the completion of first nucleotide strand (referred to as the Watson strand) passing through the same nanopore. This technology for sequentially sequencing both nucleotide strands of a double-strand DNA in the same nanopore is referred to 1D² or 2D sequencing. At block 604, double-stranded DNA molecules are provided. At block 608, a double-stranded DNA molecule is ligated with sequencing adaptors, being suitable for nanopore sequencing. At block 612, a strand of a single double-stranded molecule moved through a pore embedded in a membrane, followed by the complementary strand. At block 616, electrical current signals are obtained for both strands of each double-stranded DNA molecule. The ionic current signals could be measured by a trans-electrode. The obtained electrical currents signals were used for deducing the nucleotide information of a DNA molecule that was sequenced (i.e., base calling), using Guppy (Oxford Nanopore Technologies Ltd). In some embodiments, the other base calling tools could be used, including but not limited to Albacore (nanoporetech.com/), WaveNano (Wang et al. Quantitative Biology. 2018; 6:359-368), Chiron (Teng et al. GigaScience. 2018; 7:giy037), Flappie (github.com/nanoporetech/flappie), Scrappie (github.com/nanoporetech/scrappie), etc.

The electrical current signals which were sampled at a particular rate in time (e.g., a millisecond) would be assigned to different detected nucleotides for base modification analysis. The current signals would be processed by a segmentation step using, for example, Tombo (Stoiber et al bioRxiv. 2016; doi.org/10.1101/094672). These segmented electrical events would be assigned to different nucleotides. At block 620, an integrated presentation matrix (IPM) is constructed including both strands from each double-stranded DNA molecule. In one embodiment, the segmented electrical events associated with a nucleotide were described by a signal feature vector, i.e., [X1, X2, X3, X4, X5]. The signal feature vector from the corresponding base of the complementary strand was obtained, i.e., [X1′, X2′, X3′, X4′, X5′]. A cytosine within a CpG site and the, for example, 10-nt upstream and downstream of that cytosine (i.e., for example, a total of 21 nt), with a number of signal feature vectors, were used to form an IPM of current signal patterns. The IPM from the corresponding bases in the complementary strand of the same double-stranded DNA molecule was obtained. The IPMs derived from the Watson and Crick strands were combined, forming a new IPM matrix with a higher dimension, for base modification analysis.

In some embodiments, other computational tools could be used for assigning the electrical current signals to different nucleotides, including NanoMod (Liu et al. BMC Genomics. 2019; 20:78), Albacore (nanoporetech.com/), Chiron (Teng et al. GigaScience. 2018; 7:giy037), Nanopolish (Simpson et al. Nat Methods. 2017; 13:407-410), Scrappie (https://github.com/nanoporetech/scrappie), UNCALLED (Kovaka et al. Nat Biotechnol. 2020; doi:10.1038/s41587-020-0731-9), etc. These computational tools and other techniques described for double-stranded analysis may be used for single-stranded analysis.

For illustration purposes, a 21-nt sequence of 5′-T[CCATGC]CATCGTC[GATGCA]G-3′ was used as an example as the basis for IPM 624. IPM 624 may be similar to IPM 524 but including both the Watson and Crick strands. The bases in the brackets were left out (denoted by “. . . ”) for the sake of simplicity. For a position of -2 corresponding to the base of adenine (“A”) in the Watson strand, the signal feature vector associated with “A”, i.e. [X1=1.7, X2=0.29, X3=436, X4=24.2, X5=−0.3], was filled in the corresponding cells between a column of “−2” and a row of “A” in the area indicated by the “Watson strand”. For its corresponding base “T” in the complementary strand (i.e. the Crick strand), the signal feature vector associated with “T”, [X1′=−1.9, X2′=0.23, X3′=24.2, X4′=436, X5′=−1.4], was filled in the corresponding cells between a column of “-2” and a row of “T” in the area indicated by the “Crick strand”. The other cells in the same columns were filled by “0”. In some embodiments, the order of elements in the signal feature vector could be changed. For example, [X2, X1, X3, X4, X5], [X2, X3, X4, X5, X1], [X1, X3, X5, X4, X2] or other combinations could be used. In some embodiments, the size of the signal feature vector could be not restricted to 5. For example, the size of the signal feature vector could include, but not limited to, 6, 7, 8, 9, 10, 15, 20, 30, 40, 50, 100, etc., by adding more processed electrical signal features or raw electrical signals. The size of the signal feature vector could include, but not limited to, 1, 2, 3, 4, by editing or deleting some features in the signal feature vector.

The remaining signal feature vector for each nucleotide related to the 21-nt sequence context was filled using the same rule, thus forming a 21-nt IPM. Hence, such an IPM would simultaneously encode the current signal patterns, sequencing context, sequencing positions as well as the patterns changing over time. A number of IPMs originating from methylated and unmethylated DNA datasets were used for training a CNN or RNN model that was subsequently used to determine methylation status at a CpG site in test samples.

Block 628 shows CNN analysis. In embodiments, the CNN model made use of four two-dimensional (2D)-convolutional layers, each having 32, 64, 128, 256 filters with a kernel size of 1×25. The activation function of the rectified linear unit (ReLU) was used for those convolutional layers. A batch normalization layer was applied subsequently. A flattened layer was further added, followed by a dropout layer with a dropout rate of 0.5 and then followed by a fully connected layer comprising 200 neurons with the use of ReLU activation function. The output layer with one neuron was finally applied, with a sigmoid activation function to yield the probabilistic score for a CpG site of being methylated (i.e. probability of methylation). The program for the CNN model was implemented on the basis of the Keras deep learning framework (keras.io/). In some embodiments, one could vary the kernel size n×m, where “n” could include but not limited to 1, 2, 3, 4, 5, 10, 15, 20, 30, 35, 40, 45, 50, 100, etc., and “m” could include but not limited to 1, 2, 3, 4, 5, 10, 15, 20, 30, 35, 40, 45, 50, 100, etc.

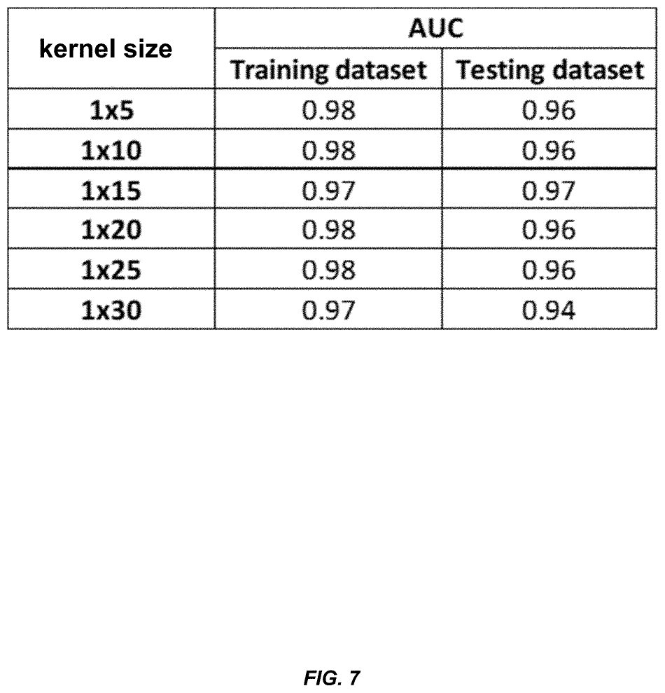

FIG. 7 is a table of the impact of kernel size on the performance of base modification analysis. The first column shows different kernel sizes. The second column shows the AUC (area under the ROC [receiver operator characteristic] curve) from the training dataset. The third column shows the AUC from the testing dataset. FIG. 7 shows that a range of kernel sizes such as 1×5, 1×10, 1×15, 1×20, and 1×25 would give a comparable performance in differentiating between methylated CpG sites and unmethylated CpG sites, as indicated by AUCs of 0.96, 0.96, 0.97, 0.96, and 0.96, respectively.

Block 632 shows RNN analysis. In embodiments, the RNN model with LSTM units was used with two full connection hidden layers, each having 256 hidden nodes. The current output of a LSTM hidden unit is determined by the current input and previous information stored in LSTM cell. As one example, the signal feature vectors, [X1, X2, X3, X4, X5], associated with a position indicating at the first row of a 21-nt IPM was considered as the input, Xₜ, for a LSTM unit, at a particular time step. A forward LSTM RNN will recursively calculate the hidden layers H according to the time steps based on the operations as follows (Gers et al. IEEE Transactions on Neural Networks. 2001; 12:1333-1340):

A_{t,F}=sigmoid (W_{xa,F}X_{t,F}+W_{ha,F}H_{t-1,F}+W_{ca,F}⊙C_{t-1,F}+b_{a,F}),

F_{t,F}=sigmoid (W_{xf,F}X_{t,F}+W_{hf,F}H_{t-1,F}+W_{cf,F}⊙C_{t-1,F}+b_{f,F}),

C_{t,F}=F_{t,F}⊙C_{t-1,F}+A_{t,F}⊙ tanh(W_{xc,F}X_{t,F}+W_{hc,F}⊙C_{t,F}+b_{c,F}),

O_{t,F}=sigmoid (W_{xo,F}X_{t,F}+W_{ho,F}H_{t-1,F}+W_{co,F}⊙C_{t,F}+b_{o,F}),

H_{t,F}=O_{t,F}⊙ tanh(C_{t,F}).

A backward LSTM RNN will recursively calculate the hidden layers H according to the time steps based on the operations as follows (Gers et al. IEEE Transactions on Neural Networks. 2001; 12:1333-1340):

A_{t,B}=sigmoid (W_{xa,B}X_{t,B}+W_{ha,B}H_{t-1,B}+W_{ca,B}⊙C_{t-1,B}+b_{a,B}),

F_{t,B}=sigmoid (W_{xf,B}X_{t,B}+W_{hf,B}H_{t-1,B}+W_{cf,B}⊙C_{t-1,B}+b_{f,B}),

C_{t,B}=F_{t,F}⊙C_{t-1,B}+A_{t,B}⊙ tanh(W_{xc,F}x_{t,B}+W_{hc,B}⊙C_{t,B}+b_{c,B}),

O_{t,}=sigmoid (W_{xo,B}X_{t,B}+W_{ho,B}H_{t-1,B}+W_{co,B}⊙C_{t,B}+b_{o,B}),

H_{t,B}=O_{t,B}⊙ tanh(C_{t,B}).

where W and b are weights and biases; X is the input vector; A is the activation vector of input gate; F is the sigmoid function of forget gate; C is the cell state; O is the sigmoid function of output gate and H is the output of the LSTM hidden units.

The outputs of forward and backward LSTM RNN units are combined.

Zₜ=H_{t,F}⊕H_{t,B}.

The last layer of LSTM RNN output was followed by a dropout layer with a dropout rate 0.2. The output layer with one neuron was finally applied, with a sigmoid activation function to yield the probabilistic score for a CpG site of being methylated (i.e., probability of methylation). The program for the CNN model was implemented on the basis of the Keras deep learning framework (keras.io/).

D. Parameter Analysis

The effect of the different electrical current vector parameters and different window sizes on AUC (area under the ROC [receiver operator characteristic] curve) is analyzed. We analyzed the differentiation power with the use of different parameters in IPM based on IPM-CNN model according to the embodiments present in this disclosure. To this end, 8,282 molecules (38,238 CpG sites) and 8,247 molecules (39,708 CpG sites) were analyzed from WGA DNA and M.SssI-treated DNA datasets, respectively.

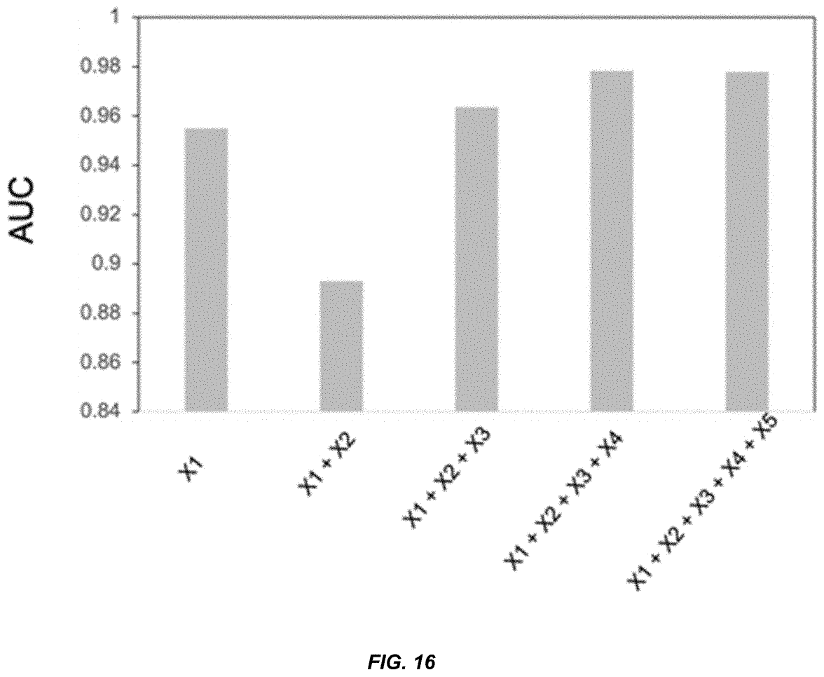

FIG. 16 shows a graph of the effect of different combinations of parameters on AUC. The different combinations of electrical current vector parameters are on the x-axis, and the AUC is on the y-axis. FIG. 16 shows that the use of a different combination of parameters of, but not limited to, X1, X2, X3, X4, and X5 in IPM led to a different performance of CpG methylation analysis. For example, the use of X1 in IPM resulted in an AUC of 0.954, whereas the combination of X1 and X2 in IPM gave rise to an AUC of 0.893. The combination of X1, X2, and X3 in IPM raised the AUC to 0.963. The combination of X1, X2, X3, and X4 in IPM further raised the AUC to 0.978, followed by the performance plateau at an AUC of 0.977 with the use of X1, X2, X3, X4, and X5 in this example. Hence, in some embodiments, the different combination of parameters in IPM would allow one to determine the desired performance in differentiation between methylated and unmethylated CpG sites.

The uses of X1, X2, X3, X4, and X5 individually rather than in combination were tested. The results of using X1, X2, X3, X4, and X5 individually resulted in AUCs of 0.95, 0.92, 0.98, 0.88, and 0.95, respectively. X3 (i.e., the median of Pᵤ in a region) resulted in a high AUC of 0.98. The high AUC may be at least partly a result of the methylation differences on a full fragment level. The dataset used involved WGA (fully unmethylated) and M.SssI (fully methylated). However, in practice, fragments will not be fully methylated or fully unmethylated. The use of X3 by itself for samples that are not fully methylated or fully unmethylated may not result in as high an AUC.

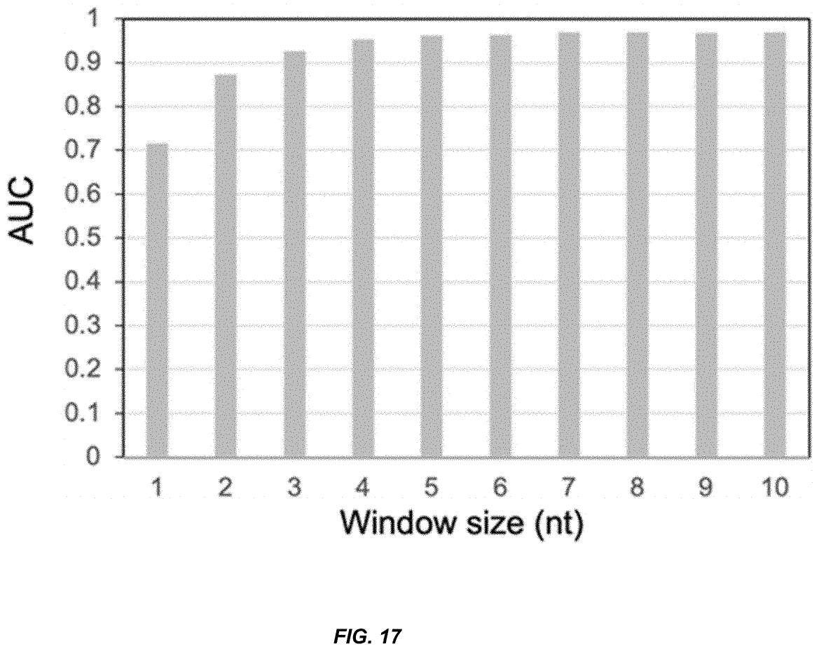

FIG. 17 shows a graph of the effect of the window size on AUC. The x-axis shows the window size in nucleotides. The y-axis shows the AUC. The number of nucleotides used in IPM (also referred to as window size) would capture different information content of current signals generated during nanopore sequencing, and may affect the performance of methylation analysis. FIG. 17 shows that the performance in differentiating between methylated and unmethylated CpG sites using IPM-CNN model appeared to gradually increase from an AUC of 0.715 to 0.969, as the number of nucleotides used in IPM increased from 1 to 10 nt. In this example, the performance plateau arrived at a window size of 7 nt. Hence, in some embodiments, adjusting the window size of IPM would allow one to determine the desired performance in differentiation between methylated and unmethylated CpG sites.

Embodiments may not require using the combination of electrical current vector parameters or window size that leads to the highest AUC. A lower AUC may be sufficient for certain uses, or a higher AUC may not be worth the additional computational and storage costs related to the additional parameters. Furthermore, different parameters may be adjusted to achieve a desired AUC, specificity, and/or sensitivity. For example, a larger window size can be used to compensate for using fewer parameters among X1, X2, X3, X4, and X5.

E. Detection of 6mA Modifications

In order to determine the applicability of electrical current signal analysis to modifications other than 5mC, the electrical current signal analysis was used to detect N6-methyladenine (6mA).

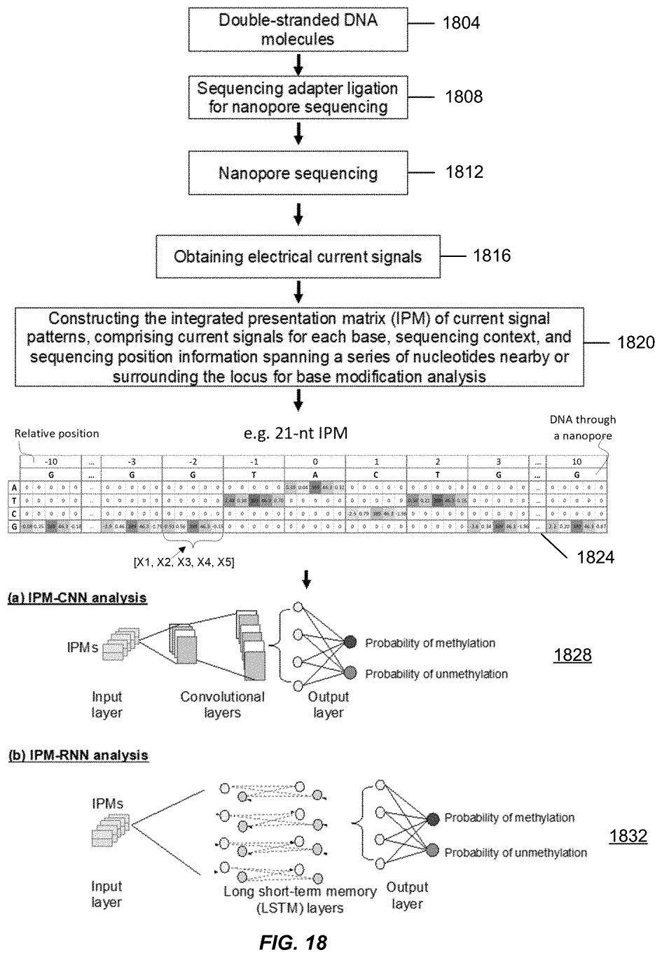

FIG. 18 shows the process for determining 6mA methylation using current signals from one strand through nanopore sequencing. FIG. 18 is similar to FIG. 5, which showed the process for determining 5mC methylation. At block 1804, double-stranded DNA molecules are provided. At block 1808, a double-stranded DNA molecule is ligated with sequencing adaptors, being suitable for nanopore sequencing. At block 1812, nanopore sequencing is performed. At block 1816, electrical current signals are obtained. At block 1820, an integrated presentation matrix (IPM) is constructed. Blocks 1804-1820 may be the same as blocks 504-520.

For illustration purposes for determining 6mA methylation, a 21-nt sequence of 5′-G[TACCCG]GGTACTG[TCTAGA]G-3′ was used as an example as the basis for IPM, centering on nucleotide A (e.g., corresponding to the position of 0) that was subject for methylation analysis. IPM 1824 shows the result of using the 21-nt sequence. The bases in the brackets were left out (denoted by “. . . ”) for the sake of simplicity. For a position of 0 corresponding to the base of adenine (“A”) in one strand, the signal feature vector associated with “A” (i.e., [X1=0.39, X2=0.04, X3=389, X4=46.3, X5=0.32]) was filled in the corresponding cells between a column of “0” and a row of “A” of the matrix. The other cells in the same columns were filled by “0”. In some embodiments, the order of elements in the signal feature vector could be changed. For example, [X2, X1, X3, X4, X5], [X2, X3, X4, X5, X1], [X1, X3, X5, X4, X2], or other combinations may be used. In some embodiments, the size of the signal feature vector may not be only 5. For example, the size of the signal feature vector could include, but not limited to, 6, 7, 8, 9, 10, 15, 20, 30, 40, 50, 100, etc., by adding more processed electrical signal features or raw electrical signals. The size of the signal feature vector could include, but not limited to, 1, 2, 3, or 4, by editing or deleting some features in the signal feature vector.

The remaining signal feature vector for each nucleotide related to the 21-nt sequence context was filled using the same rule, thus forming a 21-nt IPM. Hence, such an IPM would simultaneously encode the current signal patterns, sequencing context, sequencing positions as well as the patterns changing over time. A number of IPMs originating from methylated and unmethylated DNA datasets in relation to nucleotide A were used for training a CNN or RNN model that was subsequently used to determine methylation status at an A site in test samples. Block 1828 shows CNN analysis, and block 1832 shows RNN analysis. These blocks may be the same as blocks 528 and 532.

To test whether our approaches (IPM-CNN or IPM-RNN) illustrated above were able to determine adenine methylation (6mA), we downloaded two public datasets comprising nanopore sequencing results of pUC19 plasmid DNA from a previous study (Rand et al. Nat Methods 2017; 14:411-413). The first dataset (6mA dataset) was generated from pUC19 plasmid DNA grown in E. coli containing both dam and dcm methyltransferases, in which all GATC motifs were supposed to be methylated at A sites. The second dataset (uA dataset) was generated from DNA that was subject to PCR amplification with unmodified nucleotides, in which all A sites were supposed to be unmethylated. In the training process, we analyzed 2052 molecules containing GATC motifs from the 6mA dataset and 2081 molecules from the uA dataset using an IPM-CNN model.

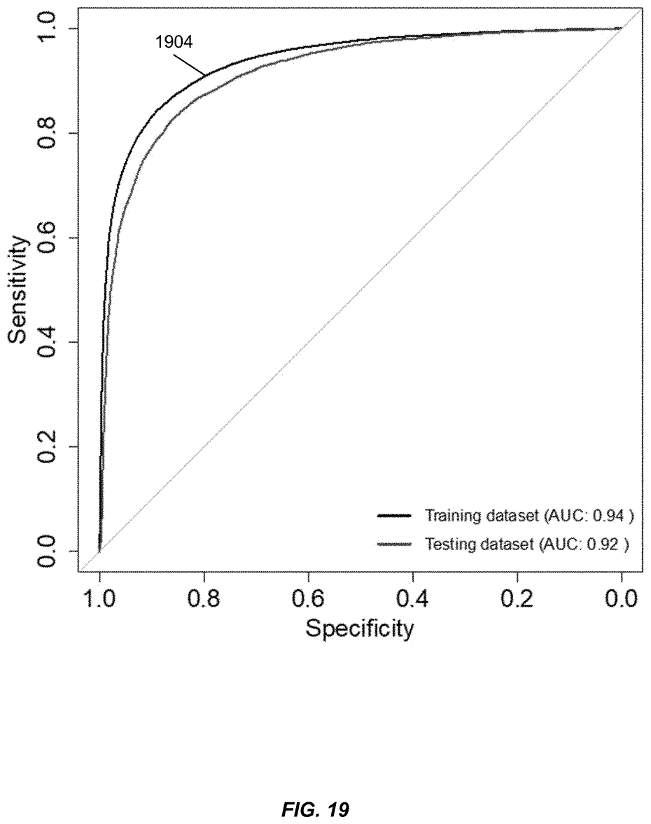

FIG. 19 shows the AUC resulting from using the IPM-CNN model. The x-axis shows the specificity. The y-axis shows the sensitivity. Line 1904 shows the results from the training dataset. The AUC with the training dataset is 0.94. In the training process, we applied the trained IPM-CNN model to 522 molecules containing GATC motifs from the 6mA dataset and 481 molecules from the uA dataset. The AUC with the testing dataset is 0.92. In addition, an AUC of 0.89 was achieved for both training and testing datasets when using a IPM-RNN model. These data suggested that IPM-CNN and IPM-RNN may allow for differentiating 6mA sites from unmethylated A sites.

In embodiments, the training datasets for 6mA determination for human or nonhuman DNA may be constructed based on PCR amplification with the use of 6mA nucleotides and unmethylated A nucleotides, respectively. After a few cycles of PCR, the majority of the DNA molecules would carry 6mA nucleotides for the dataset generated from DNA amplified with 6mA nucleotides, whereas the majority of the DNA molecules would carry unmethylated A nucleotides for the dataset generated from DNA amplified with unmethylated A nucleotides. These two types of datasets could be used for training CNN and/or RNN models for determining the methylation status of A nucleotides in a testing sample.

The use of the electrical current signal analysis to detect 6mA in addition to 5mC demonstrates the applicability of such analysis to other methylation types. Accordingly, these methods should accurately detect other methylations described herein.

F. CpG Methylation Analysis Between Non-Tumoral and Tumor Tissues of a Human Subject

Methylation of sites determined by using embodiments described herein can be used to distinguish different types of tissues. Using the IPM-RNN model according to the embodiments of the present disclosure, we analyzed the methylation patterns for cellular DNA molecules originating from nasopharyngeal carcinoma (NPC) tumor and buffy coat samples. To this end, we used 147 molecules from NPC tumor, with a median size of 4,406 bp (interquartile range (IQR): 1,962-8,128 bp) and a median of 32 CpG per molecule (IQR: 13-61). We analyzed another 147 molecules from the buffy coat with a median size of 6,823 bp (interquartile range (IQR): 2,515-9,304 bp) and a median of 49 CpG per molecule (IQR: 23-118).

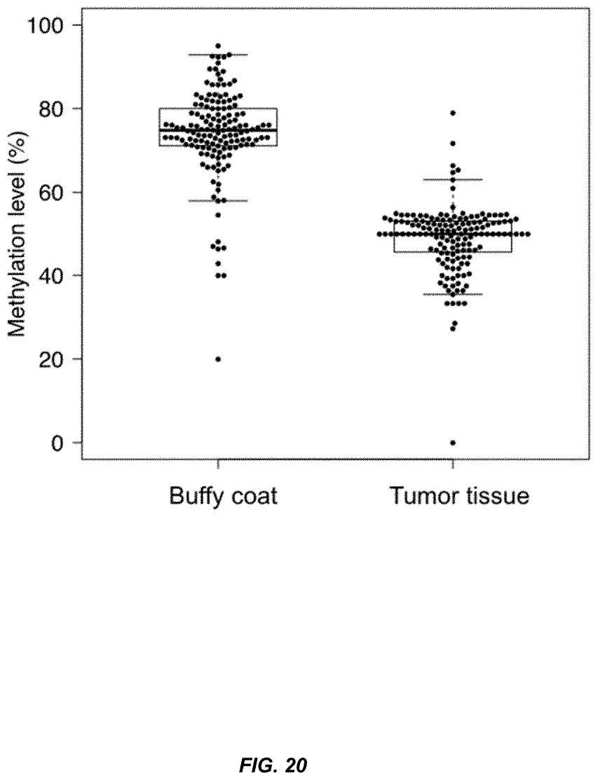

FIG. 20 shows a graph of a comparison of DNA molecules from buffy coat samples and from NPC tumor tissue samples. The x-axis shows the tissue type. The y-axis shows the methylation level as a percent. The single-molecule methylation level (i.e., the percentage of CpG sites in a molecule determined to be methylated) in the buffy coat (median: 74.8%; IQR: 71.1% to 80.1%) was found to be significantly higher than that in the NPC tumor (median: 50; IQR: 45.7 to 53.1) (P value<0.0001, Wilcoxon rank-sum test). The DNA molecules derived from tumor tissues appeared to be hypomethylated, which was consistent with a previous conclusion based on short-read bisulfite sequencing (Chan et al. Proc Natl Acad Sci USA 2013; 110:18761-8). However, the new nanopore sequencing technology described herein allows for sequencing nearly the entire long DNA molecules, and analyzing methylation patterns for DNA molecules. For example, nanopore sequencing can analyze DNA molecules greater than 600 bp in size, which are not able to be interrogated by short-read sequencing platform (e.g., Illumina).

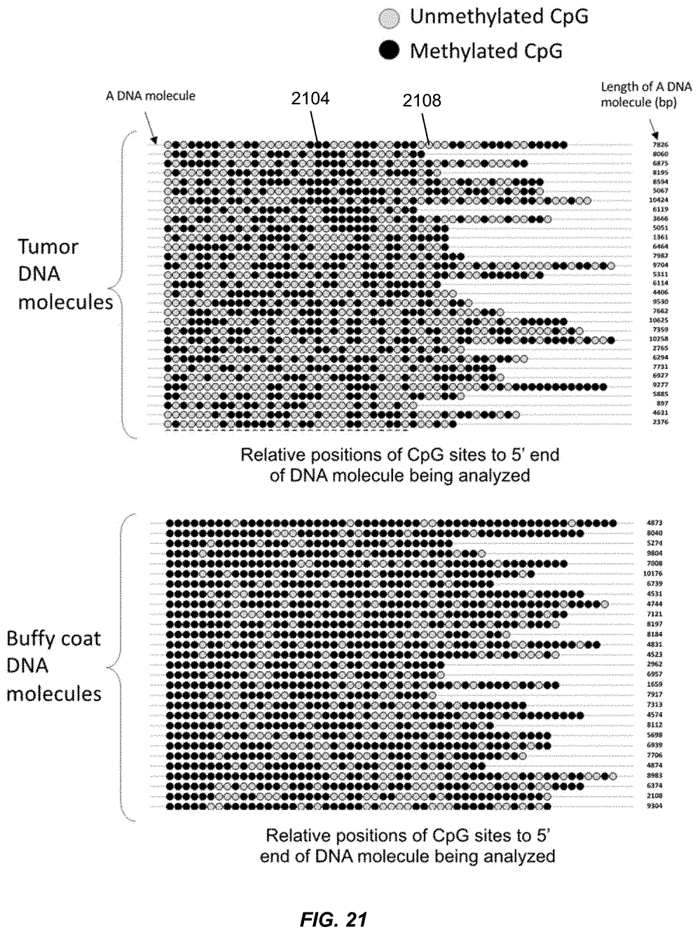

FIG. 21 illustrates the methylation patterns in tumor DNA molecules and buffy coat DNA molecules. A solid black circle (e.g., circle 2104) indicates a methylated CpG site. An unfilled circle (e.g., circle 2108) indicates an unmethylated CpG site. The circles show the relative positions of CpG sites to the 5′ end of DNA molecules (i.e., the left side of a DNA molecule in the figure is closer to the 5′ end) being analyzed. As shown in FIG. 21, the DNA molecules derived from the tumor tissue tended to carry more unmethylated CpG sites in a molecule compared with those derived from the buffy coat sample. Only 5.4% of molecules from the buffy coat sample had a single-molecule methylation level of <50% with a median length of 2,091 bp. In comparison, 39.5% of molecules from the NPC tumor tissue had a single-molecule methylation level of <50% with a median length of 2,924 bp. The lengths of DNA molecules ranged from 897 bp to 10,424 bp.

These data show that the nanopore sequencing techniques for detecting methylation described herein can be used for single-molecule methylation pattern analysis to differentiate the tissue of origin of each DNA molecule (e.g., non-tumoral DNA versus tumoral DNA molecules) from tissue biopsy samples. The analysis of single-molecule methylation pattern analysis from tissue biopsies would allow for examining tumor grades or subtypes, monitoring the treatment of cancers or other diseases, assessing the organ abnormalities (e.g., the failure of kidney), etc.

G. Analysis Between Fetal and Maternal DNA Molecules

Methylation of sites determined by using embodiments described herein can be used to distinguish between fetal and maternal DNA molecules. According to the IPM-CNN model, we determined the single-molecule methylation patterns with at least 5 CpG sites for 1,262 fetal-specific cell-free DNA molecules (median size: 530 bp; IQR: 361-779 bp) and 6,108 maternal-specific cell-free DNA molecules (median size: 668 bp; IQR: 448-1,089 bp) that were obtained from a pregnant woman at the 3^{rd} trimester, by making use of SNP information between the maternal buffy coat and placenta tissue. The fetal DNA fraction in the plasma DNA of such a pregnant woman was 26.0%.

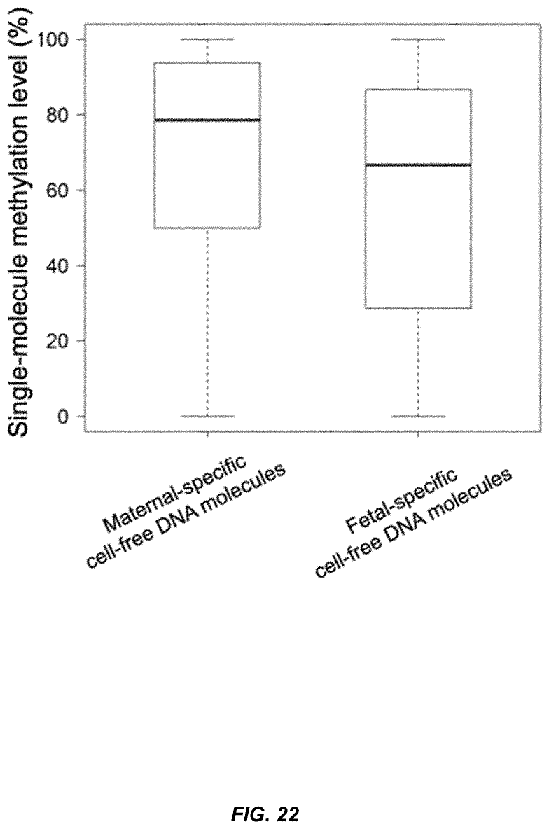

FIG. 22 shows single-molecule methylation levels between maternal-specific and fetal-specific DNA molecules. The x-axis shows the category of cell-free DNA molecules: maternal-specific or fetal-specific. The y-axis shows the single-molecule methylation level in percent. The median methylation level of a single plasma DNA molecule (i.e., the percentage of CpG sites in a molecule determined to be methylated) was 66.6% (IQR: 28.5-86.6%) for fetal-specific cell-free DNA molecules, which was significantly lower than that for maternal-specific cell-free DNA molecules (median: 78.5%; IQR: 50-93.7%) (P value: <0.0001, Mann-Whitney U test). The result suggested that the use of methylation information of cell-free DNA molecules allowed for differentiating the maternal and fetal origin of each plasma DNA molecule.

Additionally, by comparing the methylation patterns determined by an IPM-CNN model with respective reference methylation patterns of the buffy coat and placenta tissues as described in U.S. patent application Ser. No. 17/168,950, filed Feb. 5, 2021, one could achieve an AUC of 0.87 for the differentiation between plasma DNA molecules of fetal and maternal origins in a pregnant woman.

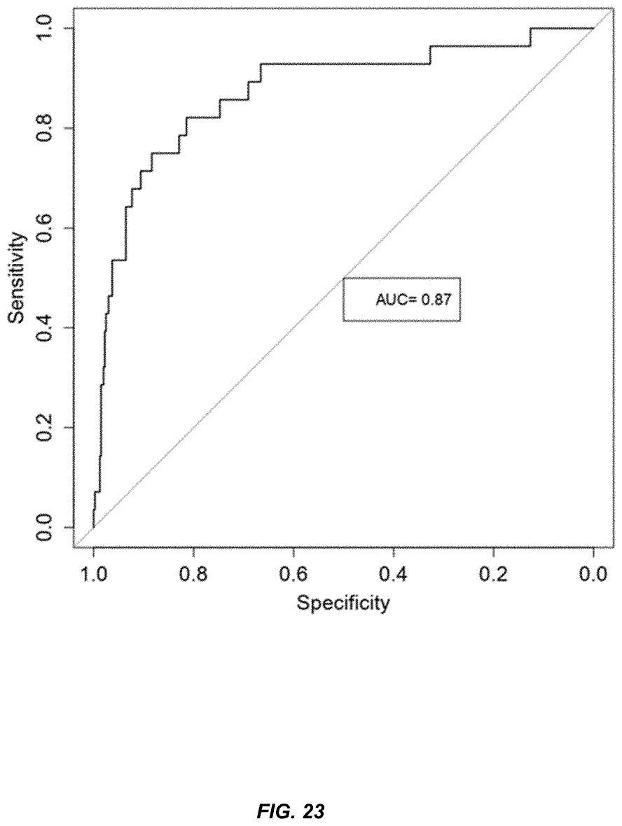

FIG. 23 shows an ROC curve for fetal and maternal origin analysis of cell-free DNA molecules in a pregnant woman on the basis of the methylation patterns determined by the IPM-CNN model. The x-axis is the specificity, and the y-axis is the sensitivity.

### III. DATASETS FOR EVALUATION OF IPM BASED METHYLATION DETERMINATION

The unmethylated dataset contained the sequencing results from amplified DNA that was prepared via whole genome amplification (WGA) (denoted as the WGA DNA dataset). The use of unmodified nucleotides in the WGA resulted in the amplified DNA containing nearly no base modifications (except for the small amount of input genomic DNA). The methylated dataset contained the sequencing results from DNA treated by the M.SssI (a CpG methyltransferase, isolated from a strain of Escherichia coli which contains the methyltransferase gene from Spiroplasma sp. strain MQ1, would methylate all CpG sites in a double-stranded DNA) prior to sequencing (denoted as the M.SssI-treated DNA dataset). M.SssI methyltransferase rendered CpG sites methylated.

For the preparation of WGA DNA dataset, the exonuclease-resistant random primer is pre-annealed to 1 ng of DNA template by incubating the reaction mix (containing phi29 reaction buffer and dNTPs) in a heat block at 95° C. for 5 min followed by cooling to 4° C. The phi29 polymerase was then added to the reaction mix and incubated at 30° C. for 4 hours. DNA was purified with Ampure XP beads and quantified with a Qubit fluorometer. Typically, 200 ng of DNA could be obtained from a 20 μ1 reaction.

For the preparation of the M.SssI-treated DNA dataset, after WGA, half of the DNA was treated with the M.SssI enzyme. Methyltransferase reaction buffer, S-adenosylmethionine (SAM) and M.SssI were mixed with DNA, and incubated at 37° C. for 2 hours. The reaction was stopped by heating at 65° C. for 20 minutes. A ligation sequencing kit (SQK-LSK109) (Oxford Nanopore) was used for library preparation. DNA was treated with NEBNext FFPE DNA Repair Mix together with NEBNext Ultra II End Repair/dA-tailing Module. After Ampure XP beads cleanup, sequencing adapters were ligated to repaired DNA by adding Adapter Mix, Ligation Buffer, and NEBNext Quick T4 DNA Ligase. Ligated DNA was cleaned up with Ampure XP beads and washed with Short Fragment Buffer. The library was resuspended in Elution Buffer. R9.4.1 flowcell was used for sequencing each of the WGA (sample_01) and M.SssI-treated (sample_02) libraries. Flowcell was first primed with a flow cell priming mix containing Flush Tether and Flush Buffer. A library loading mix was then prepared by mixing Sequencing Buffer, Loading Beads and DNA library. Library loading mix was added to the flow cell sample port in a dropwise fashion. Loaded flowcell was plugged into a slot in a PromethION and sequenced for 64 hours using default parameters.

We obtained 15.6 and 15.3 million nanopore sequencing reads for sample_01 and sample_02, respectively, among which 13.8 (88.7%) and 13.8 (90.7%) million reads could be aligned to a human reference genome (UCSC hg19) by using Minimap2 (Li H, Bioinformatics. 2018; 34(18):3094-3100). The median read length was 510 nt (interquartile range (IQR): 333-778 nt) and 606 nt (IQR: 382-911 nt) for sample_01 and sample_02, respectively. In some embodiments, BLASR (Mark J Chaisson et al, BMC Bioinformatics. 2012; 13: 238), BLAST (Altschul S F et al, J Mol Biol. 1990; 215(3):403-410), BLAT (Kent W J, Genome Res. 2002; 12(4):656-664), BWA (Li H et al, Bioinformatics. 2010; 26(5):589-595), NGMLR (Sedlazeck F J et al, Nat Methods. 2018; 15(6):461-468), and LAST (Kielbasa S M et al, Genome Res. 2011; 21(3):487-493) could be used for aligning sequenced reads to a reference genome.

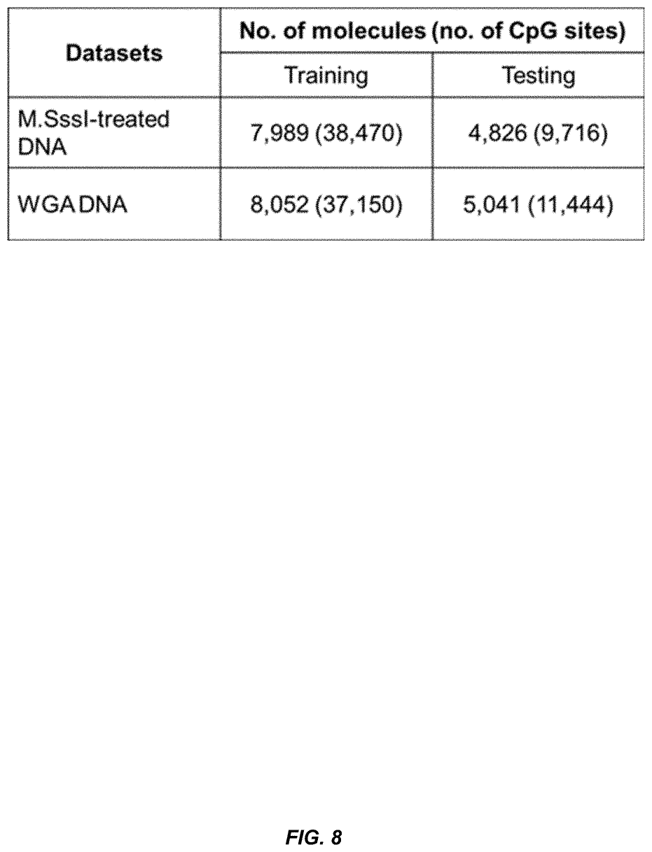

FIG. 8 is a table showing the number of sequencing molecules used for training and testing CNN and RNN models on the basis of IPMs. The first column is the dataset. M.SssI-treated DNA is methylated DNA dataset, and WGA DNA is an unmethylated DNA dataset. The second column is the number of molecules and the number of CpG sites used for training. The third column is the number of molecules and the number of CpG sites used for testing. For the training dataset, we randomly used 7,989 and 8,052 sequencing molecules from M.SssI-treated DNA (methylated DNA) and WGA DNA (unmethylated DNA), respectively. Such a training dataset comprised 38,470 methylated CpG sites and 37,150 unmethylated CpG sites. For the testing dataset, we randomly used 4,826 and 5,041 sequencing molecules from M.SssI-treated DNA (methylated DNA) and WGA DNA (unmethylated DNA), respectively. Such a training dataset comprised 9,716 methylated CpG sites and 11,444 unmethylated CpG sites.

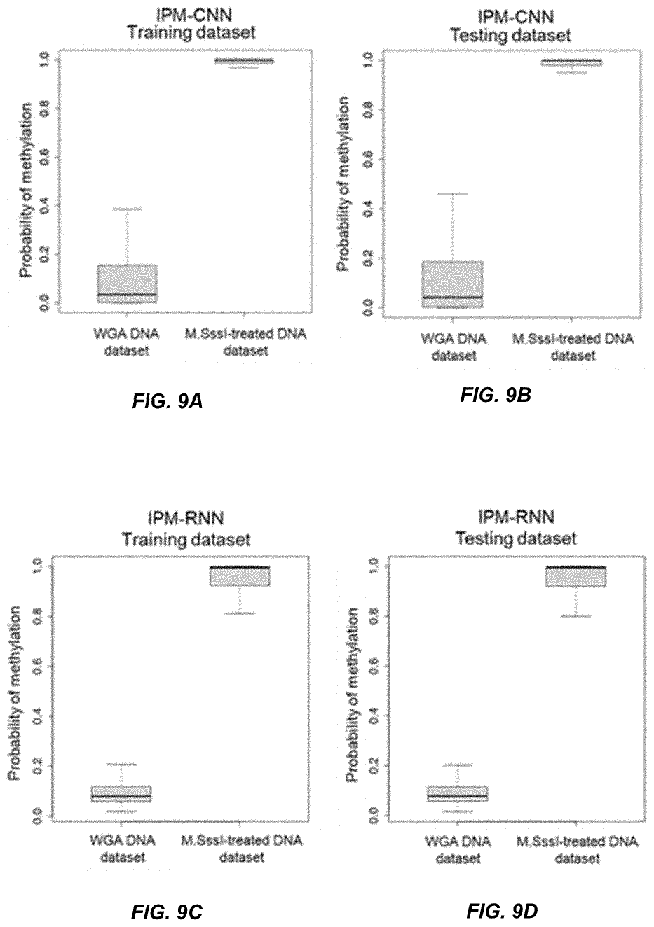

FIGS. 9A-9D are boxplots of the probability of being methylated for a CpG between WGA DNA and M.SssI-treated DNA datasets using IPM-CNN and IPM-RNN approaches. The graphs have the dataset on the x-axis. The probability of methylation is on the y-axis. FIGS. 9A and 9B show results using IPM-CNN analysis. FIG. 9A shows IPM-CNN analysis of the training dataset, where the probability of methylation for a CpG in the M.SssI-treated DNA dataset (median: 0.99; IQR: 0.987-0.999) was significantly higher than that in the WGA DNA dataset (median: 0.03; IQR: 0.001-0.15) (P value <0.0001, Mann-Whitney U test). FIG. 9B shows IPM-CNN analysis of the testing dataset, which also showed a significant difference in probability of being methylated for a CpG between the WGA (median: 0.4; IQR: 0.002-0.18) and M.SssI-treated DNA datasets (median: 0.99; IQR: 0.980-0.999) (P value<0.0001, Mann-Whitney U test).

FIGS. 9C and 9D show results using IPM-RNN analysis. FIG. 9C shows IPM-RNN analysis of the training dataset, where the probability of being methylated for a CpG in the M.SssI-treated DNA dataset (median: 0.994; IQR: 0.92-0.99) was significantly higher than that in the WGA DNA dataset (median: 0.079; IQR: 0.059-0.118) (P value<0.0001, Mann-Whitney U test). FIG. 9D shows IPM-RNN analysis of the testing dataset, which also showed a significant difference in probability of being methylated for a CpG between the WGA (median: 0.077; IQR: 0.057-0.115) and M.SssI-treated DNA datasets (median: 0.994; IQR: 0.919-0.999) (P value<0.0001, Mann-Whitney U test). These results indicated that it was feasible to use electrical signals produced by nanopore sequencing to determine the methylation status at CpG sites according to the embodiments present in this disclosure. In one embodiment, the probability of methylation cutoff of 0.5 could be used for determining the methylation status at a CpG site. With the use of this cutoff, for IPM-CNN analysis, the specificity and sensitivity for DNA methylation detection were 96% and 91% for the training dataset, respectively, and 93% and 88% for the testing dataset, respectively. For IPM-RNN analysis, the specificity and sensitivity for DNA methylation detection were 97% and 88% for both training and testing datasets, respectively. In some embodiments, the cutoff for the probability of methylation could be adjusted according to various applications.

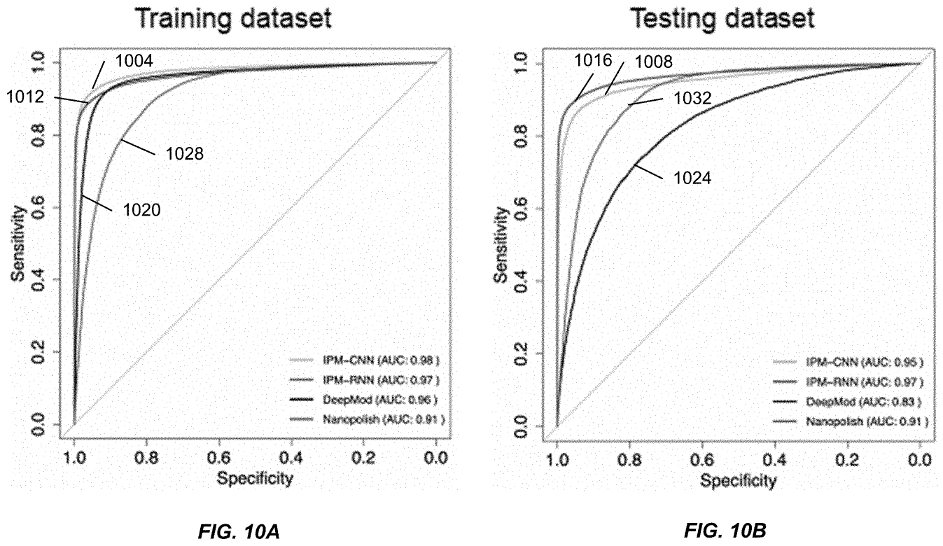

FIGS. 10A and 10B show receiver operator characteristic (ROC) curve analysis. The specificity is shown on the x-axis. The sensitivity is shown on the y-axis. FIG. 10A shows results for the training dataset. FIG. 10B shows results for the testing dataset. The IPM-CNN results are shown with lines 1004 and 1008. The IPM-RNN results are shown with lines 1012 and 1016. DeepMod (Liu et al. Nat Commun. 2019; 10:2449) results are shown with lines 1020 and 1024. Nanopolish (Liu et al. Nat Commun. 2019; 10:2449) results are shown with lines 1028 and 1032. IPM-based CNN and RNN analyses delivered good performance for both training and testing datasets, with an area under the ROC curve (AUC) of no less than 0.95. IPM-based CNN and RNN models led to a better performance with an area under the ROC curve (AUC) of 0.95 and 0.97 in the testing dataset, compared with DeepMod (0.83) and nanopolish (0.91). P values (DeLong test) for all comparisons of IPM-based RNN or CNN versus other tools including DeepMod and nanopolish were found to be <0.0001. These results indicated that IPM-CNN and IPM-RNN are superior to other tools for DNA methylation analysis.

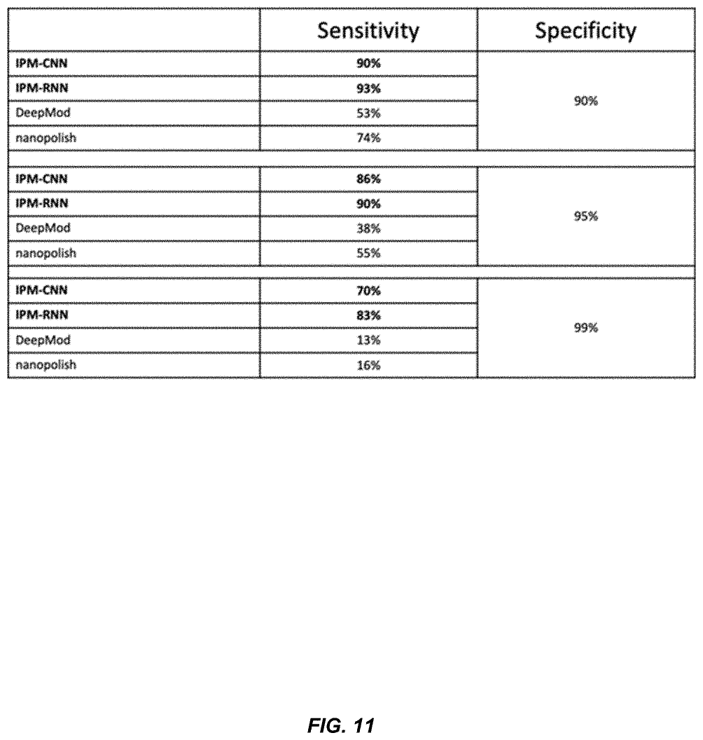

FIG. 11 is a table of sensitivities for given specificities for different analyses. The first column shows the type of analysis. The second column shows the sensitivity. The third column shows the specificity. FIG. 11 shows that with a given specificity, IPM-CNN and IPM-RNN analysis achieved much higher sensitivities. For example, with a specificity of 90%, IPM-CNN and IPM-RNN analyses achieved sensitivities of 90% and 93%, respectively, while DeepMod and nanopolish approaches achieved sensitivities of only 53% and 74%, respectively. With a specificity of 95%, IPM-CNN and IPM-RNN analyses achieved sensitivities of 86% and 90%, respectively, while DeepMod and nanopolish approaches only achieved sensitivities of 38% and 55%, respectively. With a specificity of 99%, IPM-CNN and IPM-RNN analyses achieved sensitivities of 70% and 83%, respectively, while the DeepMod and nanopolish achieved sensitivities of only 13% and 16%, respectively. These results further demonstrated that the integrated presentation matrix of current signal patterns for a sequence segment would greatly improve the accuracy of DNA methylation determination. In particular, IPM-RNN led to the best performance among those approaches.

In some embodiments, for an IPM, the length of a DNA stretch surrounding a base that was subjected to base modification analysis could be symmetrical or asymmetrical. For example, X-nt upstream and Y-nt downstream of that base could be used for base modification analysis. X could include, but is not limited to, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 300, 400, 500, 1000, 2000, 4000, 5000, and 10000; Y could include, but was not limited to, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 300, 400, 500, 1000, 2000, 4000, 5000, and 10000. X and Y may be the same or different.

In some embodiments, base modifications in nucleic acids would be analyzed according to embodiments in this disclosure across different organisms including viruses, bacteria, plants, fungi, nematodes, insects, and vertebrates (e.g., humans), etc. The most common base modifications are the addition of a methyl group to different DNA bases at different positions, so-called methylation. Methylation has been found on cytosines, adenines, thymines and guanines, such as 5mC (5-methylcytosine), 4mC (N4-methylcytosine), 5hmC (5-hydroxymethylcytosine), 5fC (5-formylcytosine), 5caC (5-carboxylcytosine), 1mA (N1-methyladenine), 3mA (N3-methyladenine), 6mA (N6-methyladenine), 7mA (N7-methyladenine), 3mC (N3-methylcytosine), 2mG (N2-methylguanine), 6mG (O6-methylguanine), 7mG (N7-methylguanine), 3mT (N3-methylthymine), and 4mT (O4-methylthymine).

In some embodiments, integrated presentation matrix of current signal patterns could be analyzed by different statistical and/or mathematical models, including, but was not limited to, linear regression, logistic regression, deep recurrent neural network (e.g., long short-term memory, LSTM), Bayes classifier, hidden Markov model (HMM), linear discriminant analysis (LDA), k-means clustering, density-based spatial clustering of applications with noise (DBSCAN), random forest algorithm, and support vector machine (SVM). In yet another embodiment, natural language processing would be applied to electrical signal analysis for base modification analysis.

In some embodiments, different types of nanopores could be used, including but not limited to biological nanopores such as the protein a-hemolysin and its variations by protein-engineering techniques, pore proteins produced by programmed bacteria, solid-state nanopores fabricated from synthetic materials, graphene, etc.

In embodiments, these methods can be used to target a large number of long DNA molecules sharing homologous sequences by designing the guide RNAs with reference to a reference genome such as a human reference genome (hg19), for example, the long interspersed nuclear element (LINE) repeats. In one example, such an analysis can be used for the analysis of circulating cell-free DNA in maternal plasma of pregnant women for the detection of fetal aneuploidies (Kinde et al. PLOS One 2012; 7(7):e41162. In embodiments, the deactivated or ‘dead’ Cas9 (dCas9) and its associated single guide RNA (sgRNA) can be used for enriching targeted long DNA without cutting the double-stranded DNA molecules. For example, the 3′ end of sgRNA could be designed to bear an extra universal short sequence. One could use biotinylated single-stranded oligonucleotides complementary to that universal short sequence to capture those target long DNA molecules bound by dCas9. In another embodiment, one could use biotinylated dCas9 protein or sgRNA, or both, to facilitate the enrichment.

In embodiments, one may perform size selection to enrich the long DNA fragments without restricting to one or more particular genomic regions of interest, using approaches including but not limited to chemical, physical, enzymatic, gel-based, and magnetic bead-based methods, or methods that combine more than such approaches.

### IV. EXAMPLE METHODS

This section shows example methods of using a machine learning model to detect a base modification and of training the machine learning model for detection of a base modification.

A. Detection of Modifications

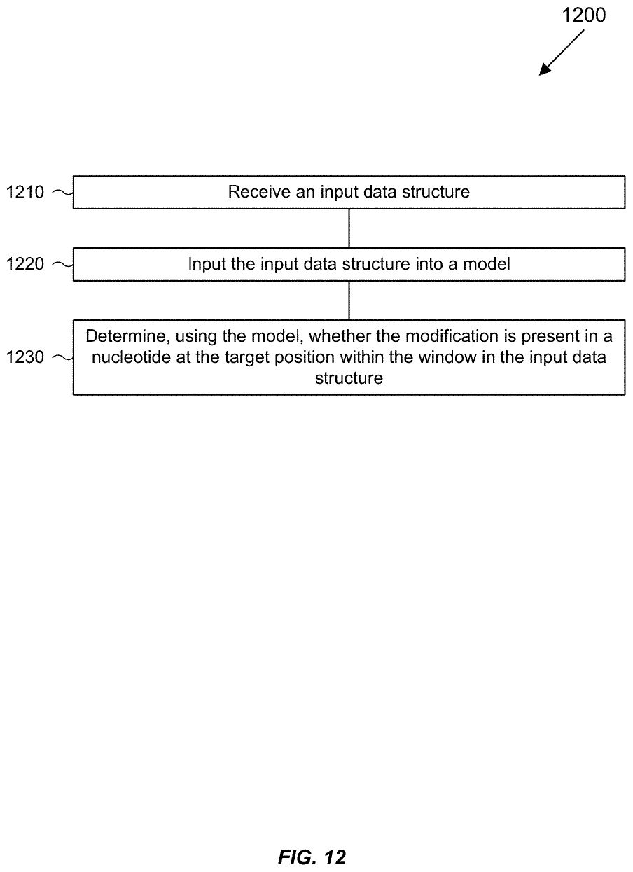

FIG. 12 is a flowchart of an example process 1200 associated with detecting a modification of a nucleotide in a nucleic acid molecule. The modification may include any methylation or any oxidation described herein. The oxidation may be 8-oxo-guanine. In some implementations, one or more process blocks of FIG. 12 may be performed by a system (e.g., measurement system 1400). In some implementations, one or more process blocks of FIG. 12 may be performed by another device or a group of devices separate from or including the system. Additionally, or alternatively, one or more process blocks of FIG. 12 may be performed by one or more components of measurement system 1400, such as detector 1420, logic system 1430, local memory 1435, external memory 1440, storage device 1445, and/or processor 1450.

At block 1210, an input data structure is received. The input data structure may correspond to a window of nucleotides sequenced in a sample nucleic acid molecule. The sample nucleic acid molecule is sequenced by measuring an electrical signal corresponding to the nucleotides. The electrical signal may be a current, voltage, resistance, inductance, capacitance, or impedance. Sequencing may be by using a nanopore. Process 1200 may further include sequencing the sample nucleic acid using a nanopore. The nanopore may be any nanopore described herein.

The input data structure may include values for several properties. Properties may include for each nucleotide within the window, an identity of the nucleotide, a position of the nucleotide with respect to a target position within the respective window, and a vector including a first segment statistical value of a segment of the electrical signal corresponding to the nucleotide. Properties may include a first region statistical value of the electrical signal in a region of the nucleic acid molecule equal to or larger than the window. For example, the input data structure may include an integrated presentation matrix [IPM].

The identity of the nucleotide may the base (e.g., A, T, C, or G). The base may be determined through base calling techniques with nanopore sequencing. The base calling techniques may associate a segment of the electrical signal with the nucleotide. The position of the nucleotide may be a nucleotide distance relative to the target position. For example, the position may be +1 when the nucleotide is one nucleotide away from the target position in one direction, and the position may be −1 when the nucleotide is one nucleotide away from the target position in the opposite direction.

The first segment statistical value may represent a mean of the segment of the electrical signal corresponding to the nucleotide. In some embodiments, the first segment statistical value may represent a variation (e.g., standard deviation) of the electrical signal of the segment of the electrical signal corresponding to the nucleotide. In embodiments, the first segment statistical value may represent a normalized value of a mean of the segment of the electrical signal corresponding to the nucleotide. Normalization may include rescaling so that the first segment statistical value is in a certain range (e.g., a range from 0 to 1). Normalization may include using the median value, the mean value, and/or deviations for part or all of the nucleotide strand. Normalization may be any described herein, including a z-score (e.g., X5).

The vector may include a second segment statistical value representing a variation of the segment of the electrical signal corresponding to the nucleotide. The vector may include a third segment statistical value representing a normalized value of the first segment statistical value. The vector may include any combination of the variables X1, X2, and X5 described herein.

The first region statistical value may represent a mean or median of the electrical signal in the region. For example, the first region statistical value may be X3. In embodiments, the first region statistical value may represent a median or mean of an absolute value of a variation of the electrical signal from the mean or median of the electrical signal in the region. The variation may be a standard deviation. For example, the first region statistical value may be X4. In some embodiments, the first region statistical value may be optional.

The input data structure may further include a second region statistical value representing a median or mean of an absolute value of a variation of the electrical signal from the mean or median of the electrical signal in the region. For example, the second region statistical value may be X4.

The first region statistical value may be the same value for different nucleotides within the window. The second region statistical value may be the same value for different nucleotides within the window. As a result, the first region statistical value and the second region statistical value may be considered separate from the vector with the first segment statistical value and/or the second segment statistical value. Alternatively, the vector may also include the first region statistical value and/or the second region statistical value may be included in the vector for each nucleotide, even though the values are the same across nucleotides. The approach to repeat the region statistical values was illustrated in IPM 524 and IPM 624.

The region may be on one strand of the sample nucleic acid molecule. In some embodiments, the region may be on two strands of the sample nucleic acid molecule. The window may include nucleotides on two strands of the sample nucleic acid molecule. The region may be the sample nucleic acid molecule. The region may include at least 5, 10, 15, 20 25, 30, 50, 100, 200, 300, 400, 500, 1 k, 5 k, 10 k, 50 k, or 1M nucleotides. In some embodiments, the region may be less than 50, 100, 200, 300, 400, 500, 1 k, 5 k, 10 k, 50 k, or 1M nucleotides. The region may be centered about the nucleotide at the target position.

The window of nucleotides may be centered about the nucleotide at the target position. In some embodiments, the window may not be centered about the nucleotide at the target position. The window can include X-nt upstream and Y-nt downstream from the nucleotide at the target position. X may include, but is not limited to, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 300, 400, 500, 1000, 2000, 4000, 5000, and 10000; Y may include, but was not limited to, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 300, 400, 500, 1000, 2000, 4000, 5000, and 10000. The minimum number of nucleotides in the window may be 2, 3, 4, 5, 6, 7, 8, 9, 10, 20, 30, 40, 50, 100, 200, or one more than the sum of any of the numbers of nucleotides upstream and downstream of the target position. The window may be similar to the window shown and described with FIG. 5.

The window may include two strands of the nucleic acid molecule, similar to the technique described with FIG. 6.

At block 1220, the input data structure is inputted into a model. The model is trained by receiving a first plurality of first data structures. Each first data structure of the first plurality of data structures corresponds to a respective window of nucleotides sequenced in a respective nucleic acid molecule of a plurality of first nucleic acid molecules. Each of the first nucleic acid molecules is sequenced by measuring the electrical signal corresponding to the nucleotides. The modification has a known first state in a nucleotide at a target position in each window of each first nucleic acid molecule. Each first data structure includes values for the same properties as the input data structure. The model may be any machine learning model described herein.

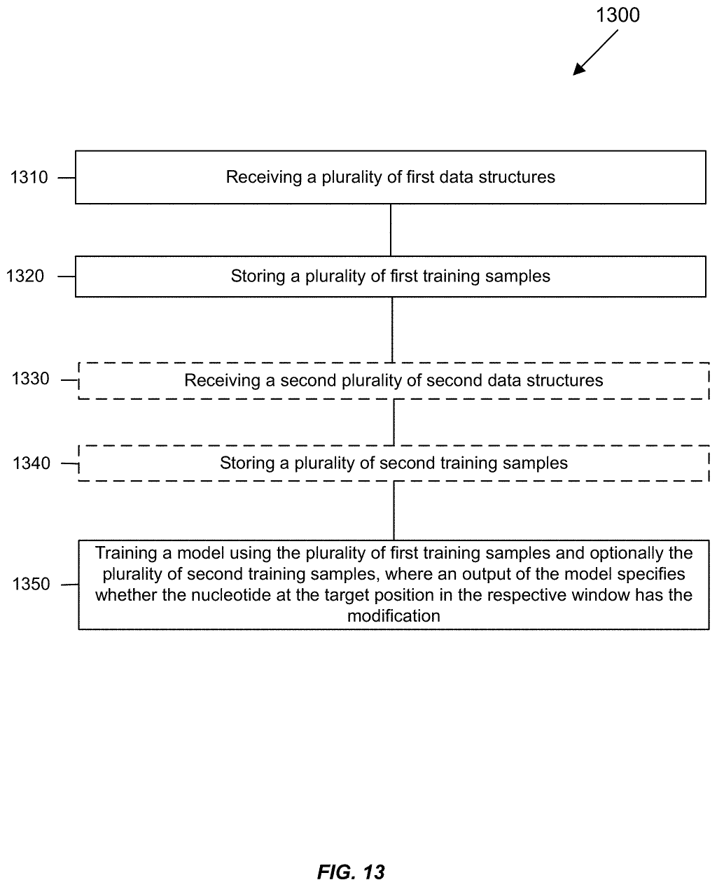

The model is further trained by storing a plurality of first training samples. Each first training sample includes one of the first plurality of first data structures and a first label indicating the first state of the nucleotide at the target position. In addition, the model is trained by optimizing, using the plurality of first training samples, parameters of the model based on outputs of the model matching or not matching corresponding labels of the first labels when the first plurality of first data structures is input to the model. An output of the model specifies whether the nucleotide at the target position in the respective window has the modification. The training may be as described later with FIG. 13.

At block 1230, the modification is determined, using the model, whether it is present in a nucleotide at the target position within the window in the input data structure.

The modification status may be used in further analysis. In a sample obtained from a pregnant woman, embodiments in this disclosure can be used to determine the fetal or maternal origin of plasma DNA molecules based on methylation status. The maternal or fetal origin may be determined by a genomic region having a higher or lower methylation level than a reference value. In embodiments, the sample obtained from a pregnant woman may be cell-free, e.g., plasma or serum. In some embodiments, the sample nucleic acid molecule may be identified as aligning to a predetermined genomic region. The predetermined genomic region may be known to be hypermethylated or hypomethylated in the fetal or maternal genome. The method may include determining the sample nucleic acid is of fetal or maternal origin using the modification status of the nucleotide at the target position and optionally the modification status of one or more other nucleotides of the sample nucleic acid molecule.

Determining whether the sample nucleic acid molecule is of fetal or maternal origin may include determining a methylation level of the sample nucleic acid molecule using the methylation statuses of the one or more nucleotides. The methylation level of the sample nucleic acid molecule may be compared to a reference value. The reference value may be determined from a methylation level of one or more maternal nucleic acid molecules. Comparing the methylation level of the sample nucleic acid molecule to the reference value may include determining the methylation level of the sample nucleic acid molecule is lower than the reference value. Determining whether the sample nucleic acid molecule is of fetal or maternal origin may include determining the sample nucleic acid molecule is of fetal origin using the comparison.

In some embodiments, the sample nucleic acid molecule may be one sample nucleic acid molecule of a plurality of sample nucleic acid molecules. The method may further include determining whether each of the plurality of sample nucleic acid molecules is fetal or maternal origin using the methylation statuses. A fetal fraction may be determined using the determination of the fetal or maternal origin of the plurality of sample nucleic acid molecules.

In some embodiments, the modification status may be used to determine whether a copy number aberration is present at a region. The modification may be a methylation. The sample nucleic acid molecule may be cell-free and obtained from a biological sample of a female subject pregnant with a fetus. The sample nucleic acid molecule may be one sample nucleic acid molecule of a plurality of sample nucleic acid molecules. The method may further include identifying the plurality of sample nucleic acid molecules as aligning to a region of a fetal genome. The modification status of one or more nucleotides of each sample nucleic acid molecule of the plurality of sample nucleic acid molecules may be determined. A methylation level of the region may be determined using the methylation statuses of the one or more nucleotides for each sample nucleic acid molecule of the plurality of sample nucleic acid molecules. The method may further include determining whether the copy number aberration is present at the region of the fetal genome using the methylation level. The region may be a chromosome, and the method may further include determining the copy number aberration is present and determining that the fetus has a chromosomal aneuploidy.

The modification may be determined to be present at one or more nucleotides. A classification of a disorder may be determined using the presence of the modification at one or more nucleotides. The classification of the disorder may include using the number of modifications. The number of modifications may be compared to a threshold. Alternatively or additionally, the classification may include the location of the one or more modifications. The location of the one or more modifications may be determined by aligning sequence reads of a nucleic acid molecule to a reference genome. The disorder may be determined if certain locations known to be correlated with the disorder are shown to have the modification. For example, a pattern of methylated sites may be compared to a reference pattern for a disorder, and the determination of the disorder may be based on the comparison. A match with the reference pattern or a substantial match (e.g., 80%, 90%, or 95% or more) with the reference pattern may indicate the disorder or a high likelihood of the disorder. The disorder may be any pregnancy-associated disorder (e.g., preeclampsia, intrauterine growth restriction, invasive placentation, and pre-term birth).

A statistically significant number of nucleic acid molecules can be analyzed so as to provide an accurate determination for a disorder, tissue origin, or clinically-relevant DNA fraction in one or more pregnant subjects. In some embodiments, at least 1,000 nucleic acid molecules are analyzed. In other embodiments, at least 10,000 or 50,000 or 100,000 or 500,000 or 1,000,000 or 5,000,000 nucleic acid molecules, or more, can be analyzed. As a further example, at least 10,000 or 50,000 or 100,000 or 500,000 or 1,000,000 or 5,000,000 sequence reads can be generated.

The method may include determining that the classification of the disorder is that the subject has the disorder. The classification may include a level of the disorder, using the number of modifications and/or the sites of the modifications.

A fetal DNA fraction, a fetal methylation profile, a maternal methylation profile, a presence of an imprinting gene region may be determined using the presence of the modification at one or more nucleotides.

Process 1200 may include additional implementations, such as any single implementation or any combination of implementations described below and/or in connection with one or more other processes described elsewhere herein.

Although FIG. 12 shows example blocks of process 1200, in some implementations, process 1200 may include additional blocks, fewer blocks, different blocks, or differently arranged blocks than those depicted in FIG. 12. Additionally, or alternatively, two or more of the blocks of process 1200 may be performed in parallel.

B. Model Training

FIG. 13 shows an example method 1300 of detecting a modification of a nucleotide in a nucleic acid molecule. Example method 1300 may be a method of training a model for detecting the modification. The modification may include a methylation. The methylation may include any methylation described herein. The modification can have discrete states, such as methylated and unmethylated, and potentially specifying a type of methylation. Thus, there may be more than two states (classifications) of a nucleotide. The training in FIG. 13 may be used with method 1200 of FIG. 12.

At block 1310, a plurality of first data structures is received. Various examples of data structures are described here, e.g., in FIGS. 5 and 6. Each first data structure of the first plurality of first data structures can correspond to a respective window of nucleotides sequenced in a respective nucleic acid molecule of a plurality of first nucleic acid molecules. Each window associated with the first plurality of data structures may include 4 or more consecutive nucleotides, including 5, 6, 7, 8, 9, 10, 11, 12, 13, 14, 15, 16, 17, 18, 19, 20, 21, or more consecutive nucleotides. Each window may have the same number of consecutive nucleotides. The windows may be overlapping. Each window may include nucleotides on a first strand of the first nucleic acid molecule and nucleotides on a second strand of the first nucleic acid molecule. The first data structure may also include for each nucleotide within the window a value of a strand property. The strand property may indicate the nucleotide being present or either the first strand or the second strand. The window may include nucleotides in the second strand that are not complementary to a nucleotide at a corresponding position in the first strand. In some embodiments, all nucleotides on the second strand are complementary to the nucleotides on the first strand. In some embodiments, each window may include nucleotides on only one strand of the first nucleic acid molecule.

The first plurality of first data structures may include 5,000 to 10,000, 10,000 to 50,000, 50,000 to 100,000, 100,000 to 200,000, 200,000 to 500,000, 500,000 to 1,000,000, or 1,000,000 or more first data structures. The plurality of first nucleic acid molecules may include at least 1,000, 10,000, 50,000, 100,000, 500,000, 1,000,000, 5,000,000, or more nucleic acid molecules. As a further example, at least 10,000 or 50,000 or 100,000 or 500,000 or 1,000,000 or 5,000,000 sequence reads can be generated.

Each of the first nucleic acid molecules is sequenced by measuring an electrical signal corresponding to the nucleotides. The electrical signal may be from nanopore sequencing.

The modification has a known first state in the nucleotide at a target position in each window of each first nucleic acid molecule. The first state may be that the modification is absent in the nucleotide or may be that the modification is present in the nucleotide. The modification may be known to absent in the first nucleic acid molecules, or the first nucleic acid molecules may undergo a treatment such that the modification is absent. The modification may be known to present in the first nucleic acid molecules, or the first nucleic acid molecules may undergo a treatment such that the modification is present. If the first state is that the modification is absent, the modification may be absent in each window of each first nucleic acid molecule and not absent only at the target position. The known first states may include a methylated state for a first portion of the first data structures and an unmethylated state for a second portion of the first data structures. The known first state for methylation may be determined through techniques using bisulfite sequencing or using optical signals from single-molecule real-time sequencing.

The target position may be the center of the respective window. For a window having spanning an even number of nucleotides, the target position may be the position immediately upstream or immediately downstream of the center of the window. In some embodiments, the target position may be at any other position of the respective window, including the first position or the last position. For example, if the window spans n nucleotides of one strand, from the 1^{st} position to the n^{th} position (either upstream or downstream), the target position may be at any from the 1^{st} position to the n^{th} position.

Each first data structure includes values for properties within the window. The properties may be any of the properties described at block 1210.

At block 1320, a plurality of first training samples is stored. Each first training sample includes one of the first plurality of first data structures and a first label indicating the first state for the modification of the nucleotide at the target position.

At block 1330, a second plurality of second data structures is received. Block 1330 is optional. Each second data structure of the second plurality of second data structures corresponds to a respective window of nucleotides sequenced in a respective nucleic acid molecule of a plurality of second nucleic acid molecules. The second plurality of nucleic acid molecules may be the same or different as the plurality of first nucleic acid molecules. The modification has a known second state in a nucleotide at a target position within each window of each second nucleic acid molecule. The second state is a different state than the first state. For example, if the first state is that the modification is present, then the second state is that the modification is absent, and vice versa. Each second data structure includes values for the same properties as the first plurality of first data structures.

At block 1340, a plurality of second training samples is stored. Block 1340 is optional. Each second training sample includes one of the second plurality of second data structures and a second label indicating the second state for the modification of the nucleotide at the target position.

At block 1350, a model is trained using the plurality of first training samples and optionally the plurality of second training samples. The training is performed by optimizing parameters of the model based on outputs of the model matching or not matching corresponding labels of the first labels and optionally the second labels when the first plurality of first data structures and optionally the second plurality of second data structures are input to the model. An output of the model specifies whether the nucleotide at the target position in the respective window has the modification. The method may include only the plurality of first training samples because the model may identify an outlier as being of a different state than the first state. The model may be a statistical model, also referred to as a machine learning model.

In some embodiments, the output of the model may include a probability of being in each of a plurality of states. The state with the highest probability can be taken as the state.

The model may include a convolutional neural network (CNN). The CNN may include a set of convolutional filters configured to filter the first plurality of data structures and optionally the second plurality of data structures. The filter may be any filter described herein. The number of filters for each layer may be from 10 to 20, 20 to 30, 30 to 40, 40 to 50, 50 to 60, 60 to 70, 70 to 80, 80 to 90, 90 to 100, 100 to 150, 150 to 200, or more. The kernel size for the filters can be 2, 3, 4, 5, 6, 7, 8, 9, 10, 11, 12, 13, 14, 15, from 15 to 20, from 20 to 30, from 30 to 40, or more. The CNN may include an input layer configured to receive the filtered first plurality of data structures and optionally the filtered second plurality of data structures. The CNN may also include a plurality of hidden layers including a plurality of nodes. The first layer of the plurality of hidden layers coupled to the input layer. The CNN may further include an output layer coupled to a last layer of the plurality of hidden layers and configured to output an output data structure. The output data structure may include the properties.

The model may include a recurrent neural network (RNN). The RNN model includes a number of Long Short-Term Memory (LSTM) units associated with the plurality of nucleotides in a measurement window. The number of LSTM units may equal the number of nucleotides in a measurement window. In some embodiments, the number of LSTM units may be less than the number of nucleotides in a measurement window. The number of LSTM units may be, but is not limited to, 1, 2, 3, 4, 5, 6, 7, 8, 9, 10, 11, 12, 13, 14, 15, 16, 17, 18, 19, 20, 21, 22, 23, 24, 25, 30, 40, 50, 100, 200, 300, 400, 500, 1,000, 2,000, 3,000, 4,000, 5,000, 10,000, 50,000, etc. One LSTM unit could transmit the information related to current signal features, which would be subjected to many rounds of linear or non-linear transformations, to a next LSTM unit. Such information transmission across LSTM units is generally organized in a sequential manner (e.g., according to the time steps). Such information transmission across LSTM units could be bidirectional (i.e., including temporal order and reserved temporal order). Each LSTM unit includes programmable operations such as forget gate, input gate, cell state, and output gate. Through those operations, one LSTM can determine whether the current signal information coming from the previous time step is to be remembered or is irrelevant and can be forgotten (forget gate). One LSTM unit tries to learn new information from the input to such a unit (input gate). The unit passes the updated information from the current time step to a next time step (output gate). The cell state herein carries the information along with all the time steps. A number of layers of LSTM units may be used. The number of LSTM layers may be 1, 2, 3, 4, 5, 6, 7, 8, 9, 10, 15, 20, 30, etc. The full connection between layers may be used. The sigmoid function is generally used as the gating function for the input, output, and forget gates. The output value of the sigmoid function may be between 0 and 1, determining either no flow or complete flow of information throughout the gates. The hyperbolic tangent activation function (also referred to as the Tanh) may be used as the output activation function which processes the information values from the output gate to form new information, with a value between −1 and 1, which may be passed to a next LSTM unit. In some embodiments, one may use other activation functions including, but not limited to, a binary step function, linear activation function, sigmoid function, rectified linear unit, etc. The values produced by the final layer of LSTM may be passed onto an output layer (i.e., dense layer, with a certain number of neurons) in which each neuron is fully connected. The number of neurons in the dense layer may be, but not limited to, 2, 3, 4, 5, 6, 7, 8, 9, 10, 20, 30, 40, 50, 100, 200, 300, 400, 500, 1000, 2000, etc. One could use a number of dense layers, including but not limited to 1, 2, 3, 4, 5, 6, 7, 8, 9, 10, 20, 30, 40, 50, 100, 5000, 1000, etc. The output layer may output the methylation score, for example based on sigmoid activation function or SoftMax activation function, which may be used for classifying methylation status. For example, if the methylation score is greater than 0.5, the base is determined to be methylated. Otherwise, the base is determined to be unmethylated. In some embodiments, the threshold used for classifying a methylated status may be, but not limited, to at least 0.1, 0.2, 0.3, 0.4, 0.6, 0.7, 0.8, 0.9, etc. In some embodiments, some of neurons in a model could be dropped out in order to minimize overfitting issues. The percentage of neurons dropped out could be but not limited to 1%, 5%, 10%, 15%, 20%, 25%, 30%, 40%, 50%, 60%, 70%, etc., which may differ depending on different layers.

The model may include a supervised learning model. Supervised learning models may include different approaches and algorithms including analytical learning, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, Nearest Neighbor Algorithm, probably approximately correct learning (PAC) learning, ripple down rules, a knowledge acquisition methodology, symbolic machine learning algorithms, subsymbolic machine learning algorithms, support vector machines, Minimum Complexity Machines (MCM), random forests, ensembles of classifiers, ordinal classification, data pre-processing, handling imbalanced datasets, statistical relational learning, or Proaftn, a multicriteria classification algorithm. The model may linear regression, logistic regression, deep recurrent neural network (e.g., long short term memory, LSTM), Bayes classifier, hidden Markov model (HMM), linear discriminant analysis (LDA), k-means clustering, density-based spatial clustering of applications with noise (DBSCAN), random forest algorithm, support vector machine (SVM), or any model described herein.

As part of training a machine learning model, the parameters of the machine learning model (such as weights, thresholds, e.g., as may be used for activation functions in neural networks, etc.) can be optimized based on the training samples (training set) to provide an optimized accuracy in classifying the modification of the nucleotide at the target position. Various form of optimization may be performed, e.g., backpropagation, empirical risk minimization, and structural risk minimization. A validation set of samples (data structure and label) can be used to validate the accuracy of the model. Cross-validation may be performed using various portions of the training set for training and validation. The model can comprise a plurality of submodels, thereby providing an ensemble model. The submodels may be weaker models that once combined provide a more accurate final model.

### V. EXAMPLE SYSTEMS

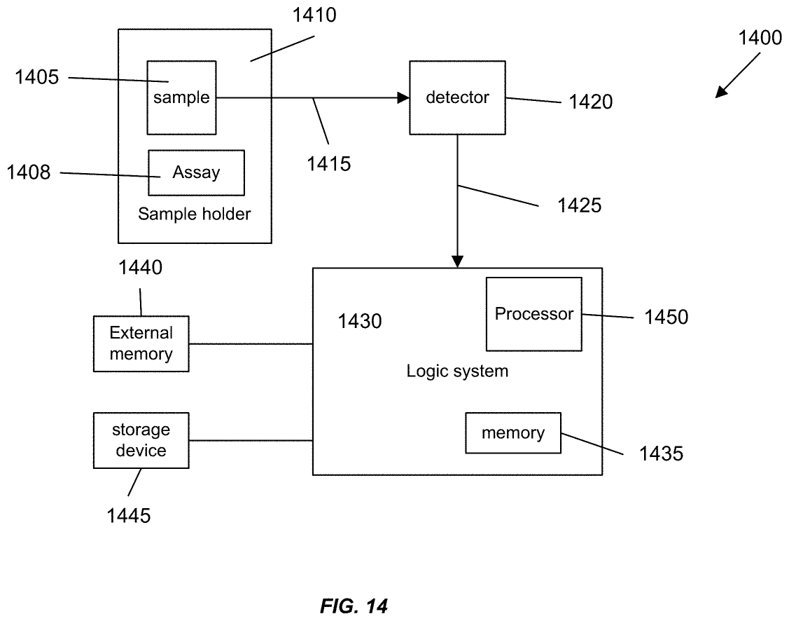

FIG. 14 illustrates a measurement system 1400 according to an embodiment of the present invention. The system as shown includes a sample 1405, such as DNA molecules within a sample holder 1410, where sample 1405 can be contacted with an assay 1408 to provide a signal of a physical characteristic 1415. An example of a sample holder can be a flow cell that includes probes and/or primers of an assay or a tube through which a droplet moves (with the droplet including the assay). Physical characteristic 1415 (e.g., a fluorescence intensity, a voltage, or a current), from the sample is detected by detector 1420. Detector 1420 can take a measurement at intervals (e.g., periodic intervals) to obtain data points that make up a data signal. In one embodiment, an analog-to-digital converter converts an analog signal from the detector into digital form at a plurality of times. Sample holder 1410 and detector 1420 can form an assay device, e.g., a sequencing device that performs sequencing according to embodiments described herein. A data signal 1425 is sent from detector 1420 to logic system 1430. Data signal 1425 may be stored in a local memory 1435, an external memory 1440, or a storage device 1445.

Logic system 1430 may be, or may include, a computer system, ASIC, microprocessor, etc. It may also include or be coupled with a display (e.g., monitor, LED display, etc.) and a user input device (e.g., mouse, keyboard, buttons, etc.). Logic system 1430 and the other components may be part of a stand-alone or network connected computer system, or they may be directly attached to or incorporated in a device (e.g., a sequencing device) that includes detector 1420 and/or sample holder 1410. Logic system 1430 may also include software that executes in a processor 1450. Logic system 1430 may include a computer readable medium storing instructions for controlling system 1400 to perform any of the methods described herein. For example, logic system 1430 can provide commands to a system that includes sample holder 1410 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

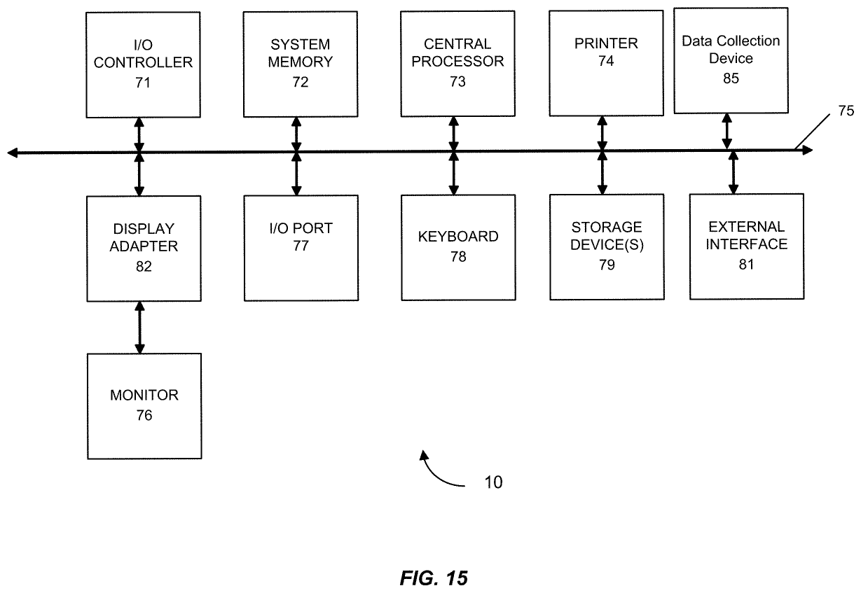

Any of the computer systems mentioned herein may utilize any suitable number of subsystems. Examples of such subsystems are shown in FIG. 15 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones, other mobile devices, and cloud-based systems.

The subsystems shown in FIG. 15 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, Lightning, Thunderbolt™). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an”, or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

All patents, patent applications, publications, and descriptions mentioned herein are incorporated by reference in their entirety for all purposes. None is admitted to be prior art.

1. A method for detecting a modification of a nucleotide in a nucleic acid molecule, the method comprising:
receiving an input data structure, the input data structure corresponding to a window of nucleotides sequenced in a sample nucleic acid molecule, wherein the sample nucleic acid molecule is sequenced by measuring an electrical signal corresponding to the nucleotides, the input data structure comprising values for the following properties:
for each nucleotide within the window:
an identity of the nucleotide,
a position of the nucleotide with respect to a target position within the respective window, and
a vector comprising a first segment statistical value of a segment of the electrical signal corresponding to the nucleotide;
inputting the input data structure into a model, the model trained by:
receiving a first plurality of first data structures, each first data structure of the first plurality of first data structures corresponding to a respective window of nucleotides sequenced in a respective nucleic acid molecule of a plurality of first nucleic acid molecules, wherein each of the first nucleic acid molecules is sequenced by measuring the electrical signal corresponding to the nucleotides, wherein the modification has a known first state in a nucleotide at a target position in each window of each first nucleic acid molecule, each first data structure comprising values for the same properties as the input data structure,
storing a plurality of first training samples, each including one of the first plurality of first data structures and a first label indicating the first state of the nucleotide at the target position, and
optimizing, using the plurality of first training samples, parameters of the model based on outputs of the model matching or not matching corresponding labels of the first labels when the first plurality of first data structures is input to the model, wherein an output of the model specifies whether the nucleotide at the target position in the respective window has the modification,
determining, using the model, whether the modification is present in a nucleotide at the target position within the window in the input data structure.

2. The method of claim 1, wherein the first segment statistical value represents a mean of the segment of the electrical signal corresponding to the nucleotide.

3. The method of claim 1, wherein the first segment statistical value represents a variation of the electrical signal of the segment of the electrical signal corresponding to the nucleotide.

4. The method of claim 1, wherein the first segment statistical value represents a normalized value of a mean of the segment of the electrical signal corresponding to the nucleotide.

5. The method of claim 1, wherein the vector comprises a second segment statistical value representing a variation of the segment of the electrical signal corresponding to the nucleotide.

6. The method of claim 1, wherein the vector comprises a second segment statistical value representing a normalized value of a mean of the segment of the electrical signal corresponding to the nucleotide.

7. The method of claim 2, wherein:
the vector comprises a second segment statistical value representing a variation of the segment of the electrical signal corresponding to the nucleotide, and
the vector comprises a third segment statistical value representing a normalized value of the first segment statistical value.

8. The method of claim 1, wherein the input data structure comprises values for a first region statistical value of the electrical signal in a region of the nucleic acid molecule equal to or larger than the window.

9. The method of claim 8, wherein the first region statistical value represents a mean or median of the electrical signal in the region.

10. The method of claim 8, wherein the first region statistical value represents a median or mean of an absolute value of a variation of the electrical signal from the mean or median of the electrical signal in the region.

11. The method of claim 9, wherein the input data structure further comprises a second region statistical value representing a median or mean of an absolute value of a variation of the electrical signal from the mean or median of the electrical signal in the region.

12. The method of claim 8, wherein the region is on one strand of the sample nucleic acid molecule.

13. The method of claim 8, wherein the region is the sample nucleic acid molecule or comprises at least 5 nucleotides.

14. The method of claim 8, wherein the region is centered about the nucleotide.

15. The method of claim 1, wherein the window comprises nucleotides on two strands of the sample nucleic acid molecule.

16. The method of claim 1, wherein the modification is a methylation or oxidation.

17. The method of claim 1, wherein the electrical signal is a current, voltage, resistance, inductance, capacitance, or impedance.

18. The method of claim 1, further comprising sequencing the sample nucleic acid molecule using a nanopore.

19. The method of claim 1, wherein:
the modification is a methylation, and
the sample nucleic acid molecule is cell-free and obtained from a biological sample of a female subject pregnant with a fetus,
the method further comprising:
determining whether the sample nucleic acid molecule is of fetal or maternal origin using a modification status of the nucleotide at the target position, wherein the modification status is whether the modification is present, and optionally the modification status of one or more other nucleotides of the sample nucleic acid molecule.

20. The method of claim 19, wherein determining whether the sample nucleic acid molecule is of fetal or maternal origin comprises:
determining a methylation level of the sample nucleic acid molecule using the modification statuses of the one or more nucleotides; and
comparing the methylation level of the sample nucleic acid molecule to a reference value.

21. The method of claim 20, wherein the reference value is determined from a methylation level of one or more maternal nucleic acid molecules.

22. The method of claim 20, wherein:
comparing the methylation level of the sample nucleic acid molecule to the reference value comprises determining the methylation level of the sample nucleic acid molecule is lower than the reference value, and
determining whether the sample nucleic acid molecule is of fetal or maternal origin comprises determining the sample nucleic acid molecule is of fetal origin using the comparison.

23. The method of claim 19, further comprising:
identifying the sample nucleic acid molecule as aligning to a predetermined genomic region.

24. The method of claim 19, wherein:
the sample nucleic acid molecule is one sample nucleic acid molecule of a plurality of sample nucleic acid molecules,
the method further comprising:
determining whether each of the plurality of sample nucleic acid molecules is fetal or maternal origin using the modification statuses, and
determining a fetal fraction using the determination of the fetal or maternal origin of the plurality of sample nucleic acid molecules.

25. The method of claim 1, wherein:
the modification is a methylation,
the sample nucleic acid molecule is cell-free and obtained from a biological sample of a female subject pregnant with a fetus, and
the sample nucleic acid molecule is one sample nucleic acid molecule of a plurality of sample nucleic acid molecules,
the method further comprising:
identifying the plurality of sample nucleic acid molecules as aligning to a region of a fetal genome,
determining a modification status of one or more nucleotides of each sample nucleic acid molecule of the plurality of sample nucleic acid molecules,
determining a methylation level of the region using the modification statuses of the one or more nucleotides for each sample nucleic acid molecule of the plurality of sample nucleic acid molecules, and
determining whether a copy number aberration is present at the region of the fetal genome using the methylation level.

26. A method for detecting a modification of a nucleotide in a nucleic acid molecule, the method comprising:
receiving a first plurality of first data structures, each first data structure of the first plurality of first data structures corresponding to a respective window of nucleotides sequenced in a respective nucleic acid molecule of a plurality of first nucleic acid molecules, wherein each of the first nucleic acid molecules is sequenced by measuring an electrical signal corresponding to the nucleotides, wherein the modification has a known first state in a nucleotide at a target position in each window of each first nucleic acid molecule, each first data structure comprising values for the following properties:
for each nucleotide within the window:
an identity of the nucleotide,
a position of the nucleotide with respect to a target position within the respective window, and
a vector comprising a first segment statistical value of a segment of the electrical signal corresponding to the nucleotide;
storing a plurality of first training samples, each including one of the first plurality of first data structures and a first label indicating the first state for the modification of the nucleotide at the target position; and
training a model using the plurality of first training samples by optimizing parameters of the model based on outputs of the model matching or not matching corresponding labels of the first labels when the first plurality of first data structures is input to the model, wherein an output of the model specifies whether the nucleotide at the target position in the respective window has the modification.

27. The method of claim 26, further comprising:
receiving a second plurality of second data structures, each second data structure of the second plurality of second data structures corresponding to a respective window of nucleotides sequenced in a respective nucleic acid molecule of a plurality of second nucleic acid molecules, wherein the modification has a known second state in a nucleotide at a target position within each window of each second nucleic acid molecule, each second data structure comprising values for the same properties as the first plurality of first data structures;
storing a plurality of second training samples, each including one of the second plurality of second data structures and a second label indicating the second state of the nucleotide at the target position;
wherein training:
the first state or the second state is that the modification is present and the other state is that the modification is absent,
the model further comprises using the plurality of second training samples by optimizing parameters of the model based on outputs of the model matching or not matching corresponding labels of the second labels when the second plurality of second data structures are input to the model.

28. The method of claim 27, wherein the plurality of first nucleic acid molecules is the same as the plurality of the second nucleic acid molecules.

29. The method of claim 26, wherein:
each window associated with the first plurality of first data structures comprises nucleotides on a first strand of the first nucleic acid molecule and nucleotides on a second strand of the first nucleic acid molecule, and
each first data structure further comprises for each nucleotide within the window a value of a strand property, the strand property indicating the nucleotide being present on either the first strand or the second strand.

30. The method of claim 26, wherein the modification comprises a methylation of the nucleotide at the target position.

31. The method of claim 30, wherein the known first states include a methylated state for a first portion of the first data structures and an unmethylated state for a second portion of the first data structures.

32-45. (canceled)

46. A computer product comprising a non-transitory computer readable medium storing a plurality of instructions that when executed control a computer system to perform the method of claim 1.

47. A system comprising:
the computer product of claim 46; and
one or more processors for executing instructions stored on the computer readable medium.

48-50. (canceled)
